(19) United States
(12) Patent Application Publication
(10) Pub. No.: US 2014/0005258 A1
(43) Pub. Date: Jan. 2, 2014

(54) Delivery of Therapeutic Agents Using Oligonucleotide-Modified Nanoparticles as Carriers

(71) Applicant: NORTHWESTERN UNIVERSITY, Evanston, IL (US)

(72) Inventors: Chad A. Mirkin, Wilmette, IL (US); David A. Giljohann, Chicago, IL (US); Weston L. Daniel, Evanston, IL (US)

(73) Assignee: NORTHWESTERN UNIVERSITY, Evanston, IL (US)

(21) Appl. No.: 14/020,081

(22) Filed: Sep. 6, 2013

Related U.S. Application Data
(63) Continuation of application No. 13/393,463, filed on Jul. 23, 2012, now abandoned.
(60) Provisional application No. 61/238,930, filed on Sep. 1, 2009.
(60) Provisional application No. 61/314,114, filed on Mar. 15, 2010.

(51) Int. Cl. A61K 47/26; A61K 31/337

(52) U.S. Cl. CPC A61K 47/26; A61K 31/337
USPC 514/449; 514/777

(57) ABSTRACT

Disclosed are drug delivery compositions comprising an oligonucleotide-modified nanoparticle and a therapeutic agent. Specifically, disclosed are compositions comprising a number of oligonucleotide molecules in a ratio to therapeutic agent molecules to allow a sufficient transportation of the therapeutic agent molecules into a cell. The therapeutic agents include both hydrophobic and hydrophilic. Different attachments of therapeutic agents in a composition are also described.

### CROSS-REFERENCE TO RELATED APPLICATIONS

This application claims the priority benefit under 35 U.S.C. §119(e) of U.S. Provisional Application No. 61/235,930, filed Sep. 1, 2009, and U.S. Provisional Application No. 61/314,114, filed Mar. 15, 2010, the disclosures of which are incorporated herein by reference in their entirety.

### STATEMENT OF GOVERNMENT INTEREST

This invention was made with government support under Grant Number 5U54 CA119341 awarded by the National Institutes of Health (NIH)/National Cancer Institute/Centers of Cancer Nanotechnology Excellence (NCI/CCNE), Grant Number CA034992, awarded by the NIH(NCI), and Grant Number 5DP1 OD000285 awarded by the National Institutes of Health (NIH). The government has certain rights in the invention.

### FIELD OF THE INVENTION

The present invention is directed to therapeutic agent delivery compositions comprising oligonucleotide-modified nanoparticles and therapeutic agents.

### BACKGROUND OF THE INVENTION

The solubility of therapeutic agents in aqueous solutions is very important for their absorption and transport to their sites of action, and is a major factor in their effectiveness as therapeutic agents and in the design of their dosage forms. Solvation of hydrophobic therapeutic agents is traditionally achieved by co-solvents, creating colloidal solutions with the therapeutic agent, emulsions and surfactants. However, each approach has associated drawbacks. The concentration of co-solvents, for instance, must be used within an acceptable degree of toxicity associated with its use, and they are typically limited to alcohol solutions. Hydrophobic therapeutic agents can be dispersed in aqueous solutions as sols on the nanometer scale. However, these dispersions typically have a very limited shelf life in solution. Therapeutic agents can be dispersed in emulsions, but this foim of delivery has not been used widely. Finally, surfactant micelles are used for the clinical delivery of therapeutic agents, but they have a number of disadvantages. For example, delivery is contingent on the therapeutic agent being released from the micelle. In addition, surfactant micelles can irritate mucous membranes and some are hemolytically active.

### SUMMARY OF THE INVENTION

Described herein is a nanoparticle composition that comprises an oligonucleotide and a therapeutic agent that is useful for intracellular delivery of the therapeutic agent. In an embodiment, a drug delivery composition is provided comprising an oligonucleotide-modified nanoparticle and a therapeutic agent, the therapeutic agent being one that is deliverable at a significantly lower level in the absence of attachment of the therapeutic agent to the oligonucleotide-modified nanoparticle compared to the delivery of the therapeutic agent when attached to the oligonucleotide-modified nanoparticle, and wherein the ratio of oligonucleotide on the oligonucleotide-modified nanoparticle to the therapeutic agent attached to the nanoparticle is sufficient to allow transport of the therapeutic agent into a cell.

In various aspects, the therapeutic agent is a low molecular weight therapeutic agent. In some embodiments, the therapeutic agent is hydrophobic. In some aspects, the therapeutic agent is hydrophilic.

In some aspects, compositions are provided that further comprise a detectable marker. In related aspects, the detectable marker is a fluorophore.

In further embodiments contemplated by the disclosure, the oligonucleotide and the therapeutic agent are independently directly attached to the nanoparticle. In various embodiments, the therapeutic agent is attached to the oligonucleotide that is attached to the nanoparticle.

In related aspects, the therapeutic agent is covalently attached to the oligonucleotide that is attached to the nanoparticle. In other aspects, the therapeutic agent is non-covalently attached to the oligonucleotide that is attached to the nanoparticle.

Embodiments contemplated by the present disclosure also include those wherein the ratio of the oligonucleotide to the therapeutic agent on a surface of the nanoparticle is at least about 1 oligonucleotide molecule:2 therapeutic agent molecules.

Compositions provided by the present disclosure also include those that further comprise an additional therapeutic agent. In some aspects, the additional therapeutic agent is attached to the oligonucleotide-modified nanoparticle. In other aspects, the additional therapeutic agent is attached to an additional oligonucleotide-modified nanoparticle. In further aspects, the additional therapeutic agent is not attached to the oligonucleotide-modified nanoparticle and freely traverses a cell membrane.

Also provided are methods of treating a disease comprising the step of administering to a mammal a therapeutically effective amount of a composition of the present disclosure.

In some embodiments, a kit is provided comprising a composition of the present disclosure.

### BRIEF DESCRIPTION OF THE DRAWINGS

FIG. 1 depicts cellular uptake of PEG-Cy5-DNA nanoconjugates (left) and PEG-Cy5 conjugates (right).

FIG. 2 depicts (A) Hydrodynamic sizes of PTX-DNA-gold nanoparticles (AuNPs), DNA-AuNPs and paclitaxel in PBS buffer (n=3). The particles or compound were suspended in PBS buffer at the equivalent paclitaxel concentration of 25 nM for dynamic light scattering (DLS) measurement; (B) TEM image of PTX-DNA@AuNPS. The scale bar is 20 nm.

FIG. 3 depicts cytotoxicity profiles of PTX-DNA-AuNPs (black triangles), paclitaxel (red squares) and compound 1 (blue circles) at the same paclitaxel dose with MCF7, SKOV-3 and MES-SA/Dx5 cells are present in the top, middle and bottom panels respectively (n=6).

FIG. 4 depicts an MTT assay of DNA-AuNPs containing equivalent oligonucleotide concentrations of 0.064, 0.32, 1.6, 8, 40, 200, 1000 nM after 48 hours incubation in MCF7 (left) and MES-SA/Dx5 (right) cells (n=6).

### DETAILED DESCRIPTION OF THE INVENTION

Oligonucleotide-functionalized nanoparticles (ON-NPs) are a unique class of conjugate consisting of a nanoparticle (NP) core that is functionalized with a shell of oligonucleotides. They are readily able to transverse cellular membranes, not requiring the addition of toxic transfection reagents. Importantly, these structures do not serve solely as vehicles for nucleic acid delivery, but exhibit cooperative properties that result from their polyvalent surfaces.

The present disclosure provides nanoparticle-based carriers for improved delivery of a therapeutic agent. Therapeutic agents contemplated are those that are able to traverse a cell membrane more effectively when attached with an oligonucleotide-functionalized nanoparticle compared to when they are not attached with an oligonucleotide-functionalized nanoparticle. Expressly excluded from the scope of the present disclosure is a nanoparticle functionalized with an oligonucleotide and a therapeutic agent that has been previously disclosed in the art.

A surprising property of ON-NPs is their ability to enter a wide variety of cell types. It has been shown in all cell types examined to date (Table 1, below) that ON-NPs can be added directly to cell culture media and are subsequently taken up by cells in high numbers. Quantification of uptake using inductively coupled plasma mass spectrometry (ICP-MS) shows that while the number of internalized particles varies as a function of cell type, concentration, and incubation time, the cellular internalization of ON-NPs is a general property of these materials. At oligonucleotide surface loadings of greater than approximately 18 pmol·cm⁻², cellular uptake can exceed one million ON-NPs per cell. The importance of the polyvalent arrangement of oligonucleotides to cellular uptake can be further emphasized when comparing ON-NPs to other types of NPs. For example, HeLa cells internalize only a few thousand citrate coated gold particles, as compared to over one million ON-NPs under nearly identical conditions. In the context of drug delivery applications, the high uptake property and high intracellular concentration of ON-NPs is extremely useful. The extraordinary uptake of ON-NPs lends itself to a method of concentrating a therapeutic agent inside cells that would take up the therapeutic agent at a reduced level in the absence of association with the ON-NP. Despite the tremendously high uptake of ON-NPs, they exhibit no toxicity in the cell types tested thus far (see Table 1, below). This property is critical for therapeutic agent delivery applications for reducing off-target effects.

TABLE 1

| Cell Type | Designation or Source |
|---|---|
| Breast | SKBR3, MDA-MB-321, AU-565 |
| Brain | U87, LN229 |
| Bladder | HT-1376, 5637, T24 |
| Colon | LS513 |
| Cervix | HeLa, SiHa |
| Skin | C166, KB, MCF, 10A |
| Kidney | MDCK |
| Blood | Sup T1, Jurkat |
| Leukemia | K562 |
| Liver | HepG2 |
| Kidney | 293T |
| Ovary | CHO |
| Macrophage | RAW 264.7 |
| Hippocampus Neurons | primary, rat |
| Astrocytes | primary, rat |
| Glial Cells | primary, rat |
| Bladder | primary, human |
| Erythrocytes | primary, mouse |
| Peripheral Blood Mononuclear Cell | primary, mouse |
| T-Cells | primary, human |
| Beta Islets | primary, mouse |
| Skin | primary, mouse |

The NP surface can act as a scaffold for the attachment of, for example, and without limitation, oligonucleotides, proteins, peptides, antibodies, antibody fragments, and small molecules. When tested in cell culture, the resultant conjugates are internalized and localized in the perinuclear region, as opposed to the cytoplasm in the case of ON-NPs. Due to their localization, these particles have an enhanced gene silencing ability (>75% decrease in target protein expression). This development is useful for drug delivery applications, as NPs can be modified with many moieties to vary the properties of the resulting conjugate. For example and without limitation, by terminating the oligonucleotides on the NP surface with N-Hydroxysuccinimide (NHS) esters, antibodies and other proteins can be covalently immobilized to the particle. These biomolecules are often leveraged for targeting of nanoparticles in vitro and in vivo, and are a useful element in an NP based drug delivery system.

it is noted here that, as used in this specification and the appended claims, the singular forms “a,” “an,” and “the” include plural reference unless the context clearly dictates otherwise.

It is further noted that the terms “attached”, “conjugated” and “functionalized” are also used interchangeably herein and refer to the association of an oligonucleotide and a therapeutic agent with a nanoparticle.

It is also noted that the term “about” as used herein is understood to mean approximately.

“Hybridization” means an interaction between two or three strands of nucleic acids by hydrogen bonds in accordance with the rules of Watson-Crick DNA complementarity, Hoogstein binding, or other sequence-specific binding known in the art. Hybridization can be performed under different stringency conditions known in the art.

### Therapeutic Agents

“Therapeutic agent,” “drug” or “active agent” as used herein means any compound useful for therapeutic or diagnostic purposes. The terms as used herein are understood to mean any compound that is administered to a patient for the treatment of a condition that can traverse a cell membrane more efficiently when attached to a nanoparticle of the disclosure than when administered in the absence of a nanoparticle of the disclosure. Therapeutic agents contemplated as part of the invention expressly exclude oligonucleotides as defined herein. Further, while it will be understood that oligonucleotides as disclosed herein may possess gene regulatory activity, this activity is not to be construed as an aspect of the present disclosure.

Therapeutic agents include but are not limited to hydrophilic and hydrophobic compounds. Accordingly, therapeutic agents contemplated by the present disclosure include without limitation drug-like molecules, proteins, peptides, antibodies, antibody fragments, aptamers and small molecules.

Protein therapeutic agents include, without limitation peptides, enzymes, structural proteins, receptors and other cellular or circulating proteins as well as fragments and derivatives thereof, the aberrant expression of which gives rise to one or more disorders. Therapeutic agents also include, as one specific embodiment, chemotherapeutic agents. Therapeutic agents also include, in various embodiments, a radioactive material.

In various aspects, protein therapeutic agents include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, hone morphogenic protein-2, bone morphogenic protein-3, bone morphogenic protein-4, hone morphogenic protein-5, bone morphogenic protein-6, bone morphogenic protein-7, hone morphogenic protein-8, bone morphogenic protein-9, bone morphogenic protein-10, hone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2α, cytokine-induced neutrophil chemotactic factor 2β,β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

The term “small molecule,” as used herein, refers to a chemical compound, for instance a peptidometic that may optionally be derivatized, or any other low molecular weight organic compound, either natural or synthetic. Such small molecules may be a therapeutically deliverable substance or may be further derivatized to facilitate delivery.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, about 1000 or more Daltons.

The term “drug-like molecule” is well known to those skilled in the art, and includes the meaning of a compound that has characteristics that make it suitable for use in medicine, for example and without limitation as the active agent in a medicament. Thus, for example and without limitation, a drug-like molecule is a molecule that is synthesized by the techniques of organic chemistry, or by techniques of molecular biology or biochemistry, and is in some aspects a small molecule as defined herein. A drug-like molecule, in various aspects, additionally exhibits features of selective interaction with a particular protein or proteins and is bioavailable and/or able to penetrate cellular membranes either alone or in combination with a composition or method of the present disclosure.

As described by the present disclosure, in some aspects therapeutic agents include small molecules (i.e., compounds having a molecular weight of less than 1000 Daltons, typically between 300 and 700 Daltons).

“Hydrophobic” as used herein is understood to mean that the solubilities in aqueous solutions for the active agents contemplated in the present disclosure are “sparingly” (30 to 100 parts solvent to dissolve 1 part solute, or active agent), “slightly” (100 to 1000 parts solvent to dissolve 1 part solute), “very slightly” (1000 to 10,000 parts solvent to dissolve 1 part solute) soluble, or “practically insoluble” (more that 10,000 parts solvent to dissolve 1 part solute) [see, e.g., The United States Pharmacopeia (USP 24/NF 19), United States Pharmacopeial Convention, Inc., 2000, incorporated by reference herein in its entirety]. The present disclosure also contemplates drugs of such a solubility that is higher than the foregoing, but that at the desired dosage would require or benefit from the assistance of a solubilizer to deliver the drug from the dosage unit in a solubilized state at a desired rate and in the desired profile. Typically, such drugs would include those that may have moderate to high solubilities, but which require a high drug load. “High drug load” as used herein means that the dosage unit contains 30% or more of the drug, where a dosage unit is the amount of a drug that is associated with a nanoparticle.

In various embodiments, therapeutic agents described in U.S. Pat. No. 7,667,004 (incorporated by reference herein in its entirety) are contemplated for use in the compositions and methods disclosed herein and include, but are not limited to, alkylating agents, antibiotic agents, antimetabolic agents, hormonal agents, plant-derived agents, and biologic agents.

Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. cat mustine, lomustine, streptozocin), nonclassic alkylating agents (altretamine, dacarbazine, and procarbazine), platinum compounds (e.g., carboplastin and cisplatin).

Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin.

Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, imatinib mesylate (or GLEEVEC®), and gemcitabine.

Examples of hormonal agents include, but are not limited to, synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), camptothecin compounds (e.g., 20(S) camptothecin, topotecan, rubitecan, and irinotecan), taxanes paclitaxel and docetaxel).

Examples of biologic agents include, but are not limited to, immuno-modulating proteins such as cytokines, monoclonal antibodies against tumor antigens, tumor suppressor genes, and cancer vaccines. Examples of interleukins that may be used in conjunction with the compositions and methods of the present invention include, but are not limited to, interleukin 2 (IL-2), and interleukin 4 (IL-4), interleukin 12 (TL-12). Examples of interferons that may be used in conjunction with the compositions and methods of the present invention include, but are not limited to, interferon α, interferon β and interferon γ. Examples of cytokines include, but are not limited to erythropoietin (epoietin α), granulocyte-CSF (filgrastin), and granulocyte, macrophage-CSF (sargramostim). Other immuno-modulating agents other than cytokines include, but are not limited to bacillus Calmette-Guerin, levamisole, and octreotide.

Further, the term therapeutic agent can, in various aspects, encompass one or more of such compounds, or one or more of such compounds in composition with any other active agent(s). Specifically excluded from the scope of the term “therapeutic agent” are oligonucleotides as described herein. Compositions and methods disclosed herein, in various embodiments, are provided wherein said nanoparticle comprises a multiplicity of therapeutic agents. In one aspect, compositions and methods are provided wherein the multiplicity of therapeutic agents are specifically attached to one nanoparticle. In another aspect, the multiplicity of therapeutic agents are specifically attached to more than one nanoparticle.

Chemotherapeutic agents contemplated for use include, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; ho tones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

Therapeutic agents useful in the materials and methods of the present disclosure can be determined by one of ordinary skill in the art. For example and without limitation, and as exemplified herein, one can perform a routine in vitro test to determine whether a therapeutic agent is able to traverse the cell membrane of a cell more effectively when attached to an oligonucleotide-functionalized nanoparticle than in the absence of attachment to the oligonucleotide-functionalized nanoparticle.

In one embodiment, methods and compositions are provided wherein a therapeutic agent is able to traverse a cell membrane about 1% more efficiently when attached to an oligonucleotide-functionalized nanoparticle than when it is not attached to the oligonucleotide-functionalized nanoparticle. In various aspects, a therapeutic agent that is able to traverse a cell membrane about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold or about 100-fold or more efficiently when attached to an oligonucleotide-functionalized nanoparticle than when it is not attached to the oligonucleotide-functionalized nanoparticle.

In another embodiment, methods and compositions are provided wherein a therapeutic agent is able to traverse a cell membrane about 1% less efficiently when attached to an oligonucleotide-functionalized nanoparticle than when it is not attached to the oligonucleotide-functionalized nanoparticle. In various aspects, a therapeutic agent that is able to traverse a cell membrane about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold or about 100-fold or less efficiently when attached to an oligonucleotide-functionalized nanoparticle than when it is not attached to the oligonucleotide-functionalized nanoparticle.

In various embodiments, a drug delivery composition is provided comprising an oligonucleotide-modified nanoparticle and a therapeutic agent, the therapeutic agent being one that is deliverable at a significantly lower level in the absence of attachment of the therapeutic agent to the oligonucleotide-modified nanoparticle compared to the delivery of the therapeutic agent when attached to the oligonucleotide-modified nanoparticle, and wherein the ratio of oligonucleotide on the oligonucleotide-modified nanoparticle to the therapeutic agent attached to the nanoparticle is sufficient to allow transport of the therapeutic agent into a cell. As used herein, “ratio” refers to a number comparison of oligonucleotide to therapeutic agent. For example and without limitation, a 1:1 ratio refers to there being one oligonucleotide molecule for every therapeutic agent molecule that is attached to a nanoparticle.

In some aspects, the ratio of the oligonucleotide to the therapeutic agent is at least about 1:2. In various aspects, the ratio of the oligonucleotide to the therapeutic agent on a surface of the nanoparticle is about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, about 1:32, about 1:33, about 1:34, about 1:35, about 1:36, about 1:37, about 1:38, about 1:39, about 1:40, about 1:41, about 1:42, about 1:43, about 1:44, about 1:45, about 1:46, about 1:47, about 1:48, about 1:49, about 1:50, about 1:51, about 1:52, about 1:53, about 1:54, about 1:55, about 1:56, about 1:57, about 1:58, about 1:59, about 1:60, about 1:61, about 1:62, about 1:63, about 1:64, about 1:65, about 1:66, about 1:67, about 1:68, about 1:69, about 1:70, about 1:71, about 1:72, about 1:73, about 1:74, about 1:75, about 1:76, about 1:77, about 1:78, about 1:79, about 1:80, about 1:81, about 1:82, about 1:83, about 1:84, about 1:85, about 1:86, about 1:87, about 1:88, about 1:89, about 1:90, about 1:91, about 1:92, about 1:93, about 1:94, about 1:95, about 1:96, about 1:97, about 1:98, about 1:99, at least about 1:100, at least about 1:110, at least about 1:120, at least about 1:130, at least about 1:140, at least about 1:150, at least about 1:160, at least about 1:170, at least about 1:180, at least about 1:190, at least about 1:200, at least about 1:210, at least about 1:220, at least about 1:230, at least about 1:240, at least about 1:250, at least about 1:260, at least about 1:270, at least about 1:280, at least about 1:290, at least about 1:300, at least about 1:310, at least about 1:320, at least about 1:330, at least about 1:340, at least about 1:350, at least about 1:360, at least about 1:370, at least about 1:380, at least about 1:390, at least about 1:400, at least about 1:410, at least about 1:420, at least about 1:430, at least about 1:440, at least about 1:450, at least about 1:460, at least about 1:470, at least about 1:480, at least about 1:490, at least about 1:500, at least about 1:510, at least about 1:520, at least about 1:530, at least about 1:540, at least about 1:550, at least about 1:560, at least about 1:570, at least about 1:580, at least about 1:590, at least about 1:600 at least about 1:610, at least about 1:620, at least about 1:630, at least about 1:640, at least about 1:650, at least about 1:660, at least about 1:670, at least about 1:680, at least about 1:690, at least about 1:700, at least about 1:710, at least about 1:720, at least about 1:730, at least about 1:740, at least about 1:750, at least about 1:760, at least about 1:770, at least about 1:780, at least about 1:790, at least about 1:800, at least about 1:810, at least about 1:820, at least about 1:830, at least about 1:840, at least about 1:850, at least about 1:860, at least about 1:870, at least about 1:880, at least about 1:890, at least about 1:900, at least about 1:910, at least about 1:920, at least about 1:930, at least about 1:940, at least about 1:950, at least about 1:960, at least about 1:970, at least about 1:980, at least about 1:990, at least about 1:1000, at least about 1:1500, at least about 1:2000, at least about 1:3000, at least about 1:4000, or at least about 1:5000 or greater.

In some aspects, the ratio of therapeutic agent to oligonucleotide-functionalized nanoparticle on a surface of the nanoparticle is at least about 1:2. In various aspects, the ratio of the oligonucleotide to the therapeutic agent on a surface of the nanoparticle is about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, about 1:32, about 1:33, about 1:34, about 1:35, about 1:36, about 1:37, about 1:38, about 1:39, about 1:40, about 1:41, about 1:42, about 1:43, about 1:44, about 1:45, about 1:46, about 1:47, about 1:48, about 1:49, about 1:50, about 1:51, about 1:52, about 1:53, about 1:54, about 1:55, about 1:56, about 1:57, about 1:58, about 1:59, about 1:60, about 1:61, about 1:62, about 1:63, about 1:64, about 1:65, about 1:66, about 1:67, about 1:68, about 1:69, about 1:70, about 1:71, about 1:72, about 1:73, about 1:74, about 1:75, about 1:76, about 1:77, about 1:78, about 1:79, about 1:80, about 1:81, about 1:82, about 1:83, about 1:84, about 1:85, about 1:86, about 1:87, about 1:88, about 1:89, about 1:90, about 1:91, about 1:92, about 1:93, about 1:94, about 1:95, about 1:96, about 1:97, about 1:98, about 1:99, at least about 1:100, at least about 1:110, at least about 1:120, at least about 1:130, at least about 1:140, at least about 1:150, at least about 1:160, at least about 1:170, at least about 1:180, at least about 1:190, at least about 1:200, at least about 1:210, at least about 1:220, at least about 1:230, at least about 1:240, at least about 1:250, at least about 1:260, at least about 1:270, at least about 1:280, at least about 1:290, at least about 1:300, at least about 1:310, at least about 1:320, at least about 1:330, at least about 1:340, at least about 1:350, at least about 1:360, at least about 1:370, at least about 1:380, at least about 1:390, at least about 1:400, at least about 1:410, at least about 1:420, at least about 1:430, at least about 1:440, at least about 1:450, at least about 1:460, at least about 1:470, at least about 1:480, at least about 1:490, at least about 1:500, at least about 1:510, at least about 1:520, at least about 1:530, at least about 1:540, at least about 1:550, at least about 1:560, at least about 1:570, at least about 1:580, at least about 1:590, at least about 1:600 at least about 1:610, at least about 1:620, at least about 1:630, at least about 1:640, at least about 1:650, at least about 1:660, at least about 1:670, at least about 1:680, at least about 1:690, at least about 1:700, at least about 1:710, at least about 1:720, at least about 1:730, at least about 1:740, at least about 1:750, at least about 1:760, at least about 1:770, at least about 1:780, at least about 1:790, at least about 1:800, at least about 1:810, at least about 1:820, at least about 1:830, at least about 1:840, at least about 1:850, at least about 1:860, at least about 1:870, at least about 1:880, at least about 1:890, at least about 1:900, at least about 1:910, at least about 1:920, at least about 1:930, at least about 1:940, at least about 1:950, at least about 1:960, at least about 1:970, at least about 1:980, at least about 1:990, at least about 1:1000, at least about 1:1500, at least about 1:2000, at least about 1:3000, at least about 1:4000, or at least about 1:5000 or greater.

The present disclosure is not limited to only certain active agents, but is, for example and without limitation, applicable to any therapeutic agent for which delivery is desired. Non-limiting examples of such active agents as well as hydrophobic drugs are found in U.S. Pat. No. 7,611,728, which is incorporated by reference herein in its entirety.

Additional therapeutic agents contemplated by the present disclosure include, without limitation, the therapeutic agents in Table 2, below.

| | | | |
|---|---|---|---|
| Abacavir Sulfate | Abbo-Code Index | Abciximab | Abobotulinumtoxina |
| Acamprosate Calcium | Accolate Tablets | Accutane Capsules | Acebutolol |
| | | | Hydrochloride |
| Acetadote Injection | Acetaminophen | Acetylcysteine | Acetylsalicyclic Acid |
| Achillea Millefolium | Aciphex Tablets | Acitretin | Aconitum Napellus |
| Acticin Cream | Actidose With Sorbitol | Actidose-Aqua | Actimmune |
| | Suspension | Suspension | |
| Activase I.V. | Active Calcium Tablets | Activella Tablets | Actonel Tablets |
| Actoplus Met Tablets | Actos Tablets | Acyclovir | Aczone Gel 5% |
| Adalimumab | Adcirca Tablets | Adefovir Dipivoxil | Adenocard IV Injection |
| Adenoscan | Adenosine | Adipex-P Capsules | Adipex-P Tablets |
| Advair Diskus 100/50 | Advair Diskus 250/50 | Advair Diskus 500/50 | Advate |
| Advicor Tablets | Afinitor Tablets | Aggrenox Capsules | Ala (Alpha-Linolenic |
| | | | Acid) |
| Albendazole | Albenza Tablets | Albumin (Human) | Albutein 5% Solution |
| Albutein 25% Solution | Albuterol | Albuterol Sulfate | Aldara Cream, 5% |
| Aldesleukin | Alefacept | Alendronate Sodium | Alferon N Injection |
| Alfuzosin Hydrochloride | Alimta For Injection | Aliskiren | Alitretinoin |
| Alkeran For Injection | Alkeran Tablets | Allantoin | Allegra Tablets |
| Allegra-D 12 Hour | Allegra-D 24 Hour | Allium Cepa | Allopurinol |
| Extended-Release Tablets | Extended-Release Tablets | | |
| Almotriptan Malate | Aloxi Injection | Alpha Tocopherol | Alpha-Hydroxy |
| | | Acetate | |
| Alpha₁-Proteinase | Alphagan P Ophthalmic | Alphanate | Alphanine SD |
| Inhibitor (Human) | Solution | | |
| Alprazolam | Altabax Ointment | Alteplase | Altretamine |
| Aluminum Hydroxide | Alvimopan | Amantadine | Ambien Tablets |
| | | Hydrochloride | |
| Ambien CR Tablets | Ambisome for Injection | Ambrisentan | Amerge Tablets |
| Amevive | Amicar 500 MG Tablets | Amicar 1000 MG Tablets | Amiloride Hydrochloride |
| Amino Acid Preparations | Aminobenzoate | Aminohippurate Sodium | Aminosalicyclic Acid |
| | Potassium | | |
| 4-Amino-Salicyclic Acid | 5-Amino-Salicyclic Acid | Amitiza Capsules | Amitriptyline |
| | | | Hydrochloride |
| Amlactin Moisturizing | Amlactin XL | Amlodipine Besylate | Amnesteem Capsules |
| Lotion and Cream | Moisturizing Lotion | | |
| Amoxicillin | Amoxil Capsules | Amoxil Tablets | Amphotericin B, |
| | | | Liposomal |
| Amrix Capsules | Anagrelide | Anakinra | Ananas Comosus |
| | Hydrochloride | | |
| Anaprox Tablets | Anaprox DS Tablets | Androgel | Angeliq Tablets |
| Angiomax for Injection | Animi-3 Capsules | Anthihemophilic Factor | Antihemophilic Factor |
| | | (Human) | (Recombinant) |
| Anti-Inhibitor Coagulant | Antithrombin | Antivenin (Black Widow | Anzemet Injection |
| Complex | | Spider Antivenin) | |
| Anzemet Tablets | Apidra Injection | Apidra Solostar Injection | Aplenzin Extended- |
| | | | Release Tablets |
| Appearex Tablets | Aprepitant | Apriso Capsules | Aralast NP Solvent |
| Aranesp for Injection | Arcalyst for | Argatroban | Aricept Tablets |
| | Subcataneous Injection | | |
| Aricept ODT Tablets | Arixtra Injection | Armodafinil | Arnica Montana |
| Aromasin Tablets | Arranon Injection | Arsenic Trioxide | Artemether |
| Asacol Delayed-Release | Asacol HD Delayed- | Ascorbic Acid | Asenapine |
| Tablets | Release Tablets | | |
| Asmanex Twisthaler | Asparaginase | Aspirin | Atacand Tablets |
| Atacand HCT 16-12.5 | Atacand HCT 32-12.5 | Atenolol | Atomoxetine |
| Tablets | Tablets | | Hydrochloride |
| Atopiclair Cream | Atorvastatin Calcium | Atovaquone | Atripla Tablets |
| Atripla Tablets | Atropine Sulfate | Atryn Lyophilized | Attenuvax |
| | | Powder | |
| Augmentin Tablets | Augmentin XR Extended | Authia Cream | Avalide Film-Coated |
| | Release Tablets | | Tablets |
| Avalide Tablets | Avandamet Tablets | Avandaryl Tablets | Avandia Tablets |
| Avapro Tablets | Avastin IV | Avelox I.V. | Avelox Tablets |
| Avinza Capsules | Avita Cream | Avita Gel | Avobenzone |
| Avocado Oil | Avodart Soft Gelatin | Axert Tablets | Axid Capsules |
| | Capsules | | |
| Azasite Ophthalmic | Azelaic Acid | Azilect Tablets | Azithromycin |
| Drops | | | |
| Azmacort Inhalation | Azor Tablets | Baclofen | Balsalazide Disodium |
| Aerosol | | | |
| Balsam Peru | Banzel Tablets | Basiliximab | Bayer Aspirin |
| Bayer Children's Low | BOG, Live (intravesical) | Beclomethasone | Beclomethasone |
| Dose Aspirin Regimen | | Dipropionate | Dipropionate |
| (81 MG) Chewable | | | Monohydrate |
| Cherry and Orange | | | |
| Beconase AQ Nasal | Bee Pollen | Beelith Tablets | Belladonna |
| Spray | | | |
| Belladonna Alkaloids | Bellis Perennis | Benadryl Allergy Ultratab | Benazepril Hydrochloride |
| | | Tablets | |
| Bendamustine | Bendroflumethiazide | Benefix Vials | Benicar Tablets |
| Hydrochloride | | | |
| Benicar HCT Tablets | Bentoquatam | Bentyl Capsules | Bentyl Injection |
| Bentyl Syrup | Bentyl Tablets | Benzoyl Peroxide | Benzyl Alcohol |
| Besifloxacin | Beta-Carotene | Betamethasone | Betamethasone |
| | | | Dipropionate |
| Betamethasone Valerate | Betaseron For SC | Betimol Ophthalmic | Bevacizumab |
| | Injection | Solution | |
| Bevitamel Tablets | Bexarotene | Bexxar | Biaxin Filmtab Tablets |
| Biaxin Granules | Biaxin XL Filmtab | Bicalutamide | Bicillin C-R Injectable |
| | Tablets | | |
| Bicillin L-A Injection | Bilberry | Bimatoprost | Bio-C Tablets |
| Bioflavonoids | Biotin | Bisacodyl | Bismuth Subcitrate |
| | | | Potassium |
| Bisoprolol Fumarate | Bivalirudin | Black Widow Spider | Boniva Tablets |
| | | Antivenin (Equine) | |
| Boostrix Vaccine | Boron | Bortezomib | Bosentan |
| Botox for Injection | Botulinum Toxin Type A | Brevibloc Injection | Brimonidine Tartrate |
| Bromelain | Bromocriptine Mesylate | Budesonide | Bumetanide |
| Bupropion Hydrochloride | Buspirone Hydrochloride | Busulfan | Butenafine |
| | | | Hydrochloride |
| Butorphanol Tartrate | Byetta Injection | Bystolic Tablets | Calcijex Injection |
| Calcipotriene | Calcitriol | Calcium | Calcium Ascorbate |
| Calcium Carbonate | Calcium Citrate | Calcium Pantothenate | Calendula Pantothenate |
| Caledula Officinalis | Camellia Sinensis | Campral Tablets | Canakinumab |
| Canasa Rectal | Cancidas For Injection | Candesartan Cilexetil | Capastat Sulfate for |
| Suppositories | | | Injection |
| Capecitabine | Capreomycin Sulfate | Capryloyl Glycine | Captopril |
| Carac Cream 0.5% | Carafate Suspension | Carafate Tablets | Carbamazepine |
| Carbatrol Capsules | Carbidopa | Carbolic Acid | Cardio Basics Tablets |
| Cardioessentials Capsules | Cardizem La Extended | Carica Papaya | Carotenoids |
| | Release Tablets | | |
| Carvedilol | Carvedilol Phosphate | Caspofungin Acetate | Castor Oil |
| Catapres-TTS | Cathflo Activase | Cefdinir | Cefixime |
| Ceftazidime | Ceftin Tablets | Ceftriaxone Sodium | Cefuroxime |
| Cefuroxime Axetil | Celebrex Capsules | Celecoxib | Celexa Tablets |
| Cephaelis Ipecacuanha | Certolizumbab Pegol | Cervidil Vaginal Insert | Cetirizine Hydrochloride |
| Cetrorelix Acetate | Cetrotide for Injection | Cevimeline | Chamomilla |
| | | Hydrochloride | |
| Chantix Tablets | Charcoal, Activated | Chelated Mineral Tablets | Chemet Capsules |
| Chloral Hydrate | Chlorambucil | Chlordiazepoxide | ChIorothiazide |
| Chlorothiazide Sodium | Chloroxylenol | Chlorpheniramine | Chlorpheniramine |
| | | Maleate | Polistriex |
| Chlorpropamide | Chlorthalidone | Cholecalciferol | Choline Bitartrate |
| Choriogonadotropin Alfa | Chromium | Chromium Picolinate | Chromium Polynicotinate |
| Chymotrypsin | Cialis Tablets | Cilastatin Sodium | Cilostazol |
| Cimetidine | Cimetidine | Cimzia | Cinacalcet Hydrochloride |
| | Hydrochloride | | |
| Ciprofloxacin | Ciprofloxacin | Cisatracurium Besylate | Citalopram |
| | Hydrochloride | | Hydrobromide |
| Citranatal 90 DHA | Citranatal Assure | Citranatal Harmony | Citrantal RX Tablets |
| | | Capsules | |
| Citric Acid | Cladribine | Clarinex Tablets | Clarinex Reditabs Tablets |
| Clarinex-D 12-Hour | Clarinex-D 24-Hour | Clarithromycin | Clavulanate Potassium |
| Extended-Release Tablets | Extended-Release Tablets | | |
| Clevidipine Butryate | Cleviprex | Climara Transdermal | Climara Pro Transdermal |
| | | System | System |
| Clindamycin | Clindamycin Phosphate | Clinoril Tablets | Clobetasol Propionate |
| Clofarabine | Clorlar for Intravenous | Clomipramine | Clonazepam |
| | Infusion | Hydrochloride | |
| Clonidine | Clonidine Hydrochloride | Clopidogrel Bisulfate | Clorazepate Dipotassium |
| Clorpactin WCS-90 | Clorpres Tablets | Clotrimazole | Clozapine |
| CM Plex Cream | CM Plex Softgels | Coagulation Factor VIIA, | Coartem Tablets |
| | | Recombinant | |
| Cod Liver Oil | Codeine Phosphate | Coenzyme Q-10 | Colesevelam |
| | | | Hydrochloride |
| Collagen | Collagenase | Colocynthis | Colostrum |
| Combigan Ophthalmic | Combivir Tablets | Comtan Tablets | Comvax |
| Solution | | | |
| Concept DHA Prenatal | Concept OB Prenatal | Concerta Extended- | Copaxone for Injection |
| Multivitamin | Multivitamin | Release Tablets | |
| Supplements | Supplements | | |
| Copper | Copper, Intrauterine | Coquinone 30 Capsules | Cordymax CS-4 Capsules |
| Coreg Tablets | Coreg CR Extended- | Correctol Delayed- | Cosmegen for Injection |
| | Release Capsules | Release Tablets, USP | |
| Cozaar Tablets | Creon Delayed-Release | Crestor Tablets | Crixivan Capsules |
| | Capsules | | |
| Cubicin for Injection | Cupric Oxide | Cuprimine Capsules | Cyclobenzaprine |
| | | | Hydrochloride |
| | | | Cycloserine |
| Cyclosporine | Cymbalta Delayed- | Cysteine | Cytomel Tablets |
| | Release Capsules | | |
| Dacogen Injection | Dactinomycin | D-Alpha Tocopherol | Dalteparin Sodium |
| Dapsone | Daptomycin | Daraprim Tablets | Darbepoetin Alfa |
| Darifenacin | Darvocet-A 500 Tablets | Darvocet-N 50 Tablets | Darvocet-N 100 Tablets |
| Darvon Pulvules | Darvon-N Tablets | Daytrana Transdermal | Ddrops Dietary |
| | | Patch | Supplement |
| Decitabine | Deferasirox | Delatestryl Injection | Demser Capsules |
| Denavir Cream | Denileukin Diftitox | Depakene Capsules | Depakote Delayed |
| | | | Release Tablets |
| Depakote ER Extended | Depakote Sprinkle | Deprenyl | Derma-Smoothe/FS |
| Release Tablets | Capsules | | Topical Oil |
| Dermotic Oil | Desflurane | Desloratadine | Desonide |
| Desvenlafaxine Succinate | Dexamethasone | Dexedrine Spansule | Dexlansoprazole |
| | | Sustained-Release | |
| | | Capsules | |
| Dexmethylphenidate | Dextroamphetamine | Dextromethorphan | Dextrose |
| Hydrochloride | Sulfate | Hydrobromide | |
| DHA (Docosahexaenoic | Diazepam | Diazoxide | Dibasic Sodium |
| Acid) | | | Phosphate |
| Dibenzyline Capsules | Diclofenac Epolamine | Diclofenac Potassium | Diclofenac Sodium |
| Dicyclomine | Didronel Tablets | Dietary Supplement | Digestive Enzymes |
| Hydrochloride | | | |
| Digibind for Injection | Digoxin Immune Fab | Dilaudid Injection | Dilaudid Tablets |
| Digoxin | (Ovine) | | |
| Dilaudid-HP Injection | Dilaudid-HP Lyophilized | Diltiazem Hydrochloride | Dinoprostone |
| | Powder 250 MG | | |
| Dioctyl Sodium | Diovan Tablets | Diovan HCT Tablets | Diphenhydramine |
| Sulfosuccinate | | | Hydrochloride |
| Diphenoxylate | Diphenylhydantoin | Diphtheria & Tetanus | Diphtheria and Tetanus |
| Hydrochloride | | Toxoids and Acellular | Toxoids and Acellular |
| | | Pertussis Vaccine | Pertussis Adsorbed and |
| | | Adsorbed | Inactivated Poliovirus |
| | | | Vaccine |
| Dipyridamole | Disocorea | Divalproex Sodium | Divigel |
| Divista Softgel Capsules | Docetaxel | Docosahexanenoic Acid | Docusate Sodium |
| | | (DHA) | |
| Dolasetron Mesylate | Donepezil Hydrochloride | Donnatal Extentabs | Doribax Injection |
| Dornase Alfa | Doryx Delayed-Release | Dorzolamide | Doxazosin Mesylate |
| | Tablets | Hydrochloride | |
| Doxepin Hydrochloride | Doxil Injection | Doxorubicin | Doxycycline |
| | | Hydrochloride Liposome | |
| Doxycycline Hyclate | Dronedarone | Drospirenone | Drotrecogin Alfa |
| | | | (Activated) |
| Duet Tablets | Duet DHA Tablets and | Duetact Tablets | Duloxetine |
| | Softgel Capsules | | Hydrochloride |
| Duraclon Injection | Dutasteride | Dyazide Capsules | Dynacirc CR Controlled |
| | | | Release Tablets |
| Dyrenium Capsules | Dysport for Injection | Echinacea Angustifolia | Echinacea Purpurea |
| EC-Naprosyn Delayed- | Eculizumab | Edecrin Tablets | Edecrin Sodium |
| Release Tablets | | | Intravenous |
| Edetate Calcium | E.E.S. 400 Filmtab | E.E.S. Granules | Efavirenz |
| Disodium | Tablets | | |
| Effexor XR Extended- | Effient Tablets | Effient Tablets | Eicosapentaenoic Acid |
| Release Capsules | | | (EPA) |
| Eldepryl Capsules | Elidel Cream 1% | Eligard 7.5 MG | Eligard 22.5 MG |
| Eligard 30 MG | Eligard 45 MG | Elitek | Elmiron Capsules |
| Eloxatin for Injection | Elspar for Injection | Elspar for Injection | Eltrombopag |
| Embeda Extended | Emend Capsules | Emend for Injection | Emtricitabine |
| Release Capsules | | | |
| Emtriva Capsules | Emtriva Oral Solution | Enablex Extended- | Enalapril Maleate |
| | | Release Tablets | |
| Enbrel for Injection | Enflurane | Engerix-B Vaccine | Enjuvia Tablets |
| Enoxaparin Sodium | Entacapone | Entereg Capsules | Enzymes, Collagenolytic |
| Enzymes, Debridement | Enzymes, Digestive | Enzymes, Proteolytic | Epinastine Hydrochloride |
| Epinephrine | Epipen Auto-Injector | Epipen 2-Pak | Epipen Jr. Auto-Injector |
| Epipen Jr. 2-Pak | Epivir Oral Solution | Epivir Tablets | Epivir-HBV Oral |
| | | | Solution |
| Epivir-HBV Tablets | Epoetin Alfa | Epogen for Injection | Epoprostenol Sodium |
| Eprosartan Mesylate | Eptifibatide | Epzicom Tablets | Equetro Extended- |
| | | | Release Capsules |
| Erlotinib | Ertapenem | Eryped 200 & Eryped 400 | Erthromycin |
| | | Oral Suspension | Ethylsuccinate |
| Escitalopram Oxalate | Esmolol Hydrochloride | Esomeprazole | Esomeprazole Sodium |
| | | Magnesium | |
| Entrace Tablets | Estradiol | Estradiol Acetate | Estrogens, Conjugated, |
| | | | Synthetic B |
| Estropipate | Estrostep FE Tablets | Etanercept | Ethacrynate Sodium |
| Ethacrynic Acid | Ethinyl Estradiol | Ethosuximide | Editronate Disodium |
| Etoposide | Euphrasia Officinalis | Everolimus | Evista Tablets |
| Evoxac Capsules | Exelon Capsules | Exemestane | Exenatide |
| Exforge Tablets | Exforge HCT Tablets | Exjade Tablets | Extavia Kit |
| Ez-Char Activated | Ezetimibe | Factor IX (Human) | Factor IX Complex |
| Charcoal Pellets | | | |
| Famotidine | Fanapt Tablets | Faslodex Injection | Fatty Acids |
| Febuxostat | Feiba VH | Felodipine | Femara Tablets |
| Femcon FE Tablets | Femhrt Tablets | Femtrace Tablets | Fenofibrate |
| Fenoglide Tablets | Fenoprofen Calcium | Fentanyl | Fentanyl Citrate |
| Fentora Tablets | Ferralet 90 Tablets | Ferralet 90 Tablets | Ferrous Fumarate |
| Ferrous Fluconate | Ferrous Sulfate | Fesoterodine Fumarate | Fexofenadine |
| | | | Hydrochloride |
| Fiber | Fiber Supplement | Filgrastim | Finasteride |
| Flebogamma 5% DIF | Flecainide Acetate | Fleet Babylax | Fleet Bisacodyl Laxatives |
| | | Suppositories | |
| Fleet Pedia-Lax | Flexbumin 25% I.V. | Flolan for Injection | Flonase Nasal Spray |
| Chewable Tablets | | | |
| Florical Capsules | Florical Tablets | Flovent Diskus 50 MCG | Flovent Diskus 100 MCG |
| Flovent Diskus 250 MCG | Flovent HFA 44 MCG | Flovent HFA 110 MCG | Flovent HFA 250 MCG |
| | Inhalation Aerosol | Inhalation Aerosol | Inhalation Aerosol |
| Fluarix Vaccine | Fludarabine Phosphate | Flulaval Injection | Flumazenil |
| | | Vaccine | |
| Flumist Vaccine | Fluocinolone Acetonide | Fluocinonide | Fluorouracil |
| Fluoxetine | Fluoxetine Hydrochloride | Fluphenazine | Flurazepam |
| | | Hydrochloride | Hydrochloride |
| Flurbiprofen | Fluticasone Furoate | Fluticasone Propionate | Fluvoxamine Maleate |
| Focalin XR Capsules | Folate | Folgard OS Tablets | Folic Acid |
| Follistim AQ Cartridge | Follitropin Alfa | Follitropin Beta | Fondaparinux Sodium |
| Foradil, Aerolizer | Forane Liquid for | Formadon Solution | Formaldehyde |
| | Inhalation | | |
| Formoterol Fumarate | Formoterol Fumarate | Fortaz Injection | Fortaz for Injection |
| | Dihydrate | | |
| Forteo for Injection | Fosamax Tablets | Fosamax Plus D Tablets | Fosamprenavir Calcium |
| Fosaprepitant | Foscarnet Sodium | Foscavir Injection | Fosrenol Chewable |
| Dimeglumine | | | Tablets |
| Fragmin Injection | Frova Tablets | Frovatriptan Succinate | Fulvestrant |
| Furosemide | Gabitril Tablets | Galantamine | Gammagard Liquid |
| Gammagard S/D | Gamunex | Ganoderma Lucinum | Gardasil Injection |
| | | Mushroom Extract | |
| Gemcitabine | Gemtuzumac | Gemzar for Injection | Gengraf Capsules |
| Hydrochloride | Ozogamicin | | |
| Genotropin Lyophilized | Geodon Capsules | Geodon for Injection | Glatiramer Acetate |
| Powder | | | |
| Gleevec Tablets | Gliadel Wafer | Glimepiride | Glipizide |
| Glucagon | Glucono-Delta-Lactone | Glucosamine Sulfate | Glutose 15, Glutose 45 |
| | | | (Oral Glucose Gel) |
| Glyburide | Glycerin | Glyceryl Guaiacolate | Glyceryl Trinitrate |
| Glycyrrhestinic Acid | Goldenseal | Golimumab | Gonal-F For Injection |
| Gonal-F RFf for Injection | Gonal-F RFF Pen for | Gordochom Solution | Granisetron |
| | Injection | | Hydrochloride |
| Guaifenesin | Guanfacine | Haemophilus B | Haldol Injection |
| | Hydrochloride | Conjugate Vaccine | |
| Haldol Decanoate | Haloperidol | Hamamelis Virginiana | Happycode Spray |
| Injection | | | |
| Havrix Injection Vaccine | Hemin | Hemocyte Tablets | Hemofil M |
| Hepatitis A Vaccine, | Hepatitis B Vaccine, | HEP-Forte Capsules | Heplive Softgel Capsules |
| Inactivated | Recombinant | | |
| Hepsera Tablets | Herbals, Multiple | Herbals with Minerals | Herbals with Vitamins & |
| | | | Minerals |
| Herceptin I.V. | Hexalen Capsules | Histrelin Acetate | Homeopathic |
| | | | Formulation |
| Humalog-Pen and | Humatrope Vials and | Humira Injection Syringe | Humulin 50/50, 100 |
| Kwikpen | Cartridges | and Pen | Units |
| Humulin 70/30 Vial | Humulin N Vial | Humulin R | Humulin R (U-500) |
| Hyalgan Solution | Hycamtin Capsules | Hycamtin for Injection | Hycet Oral Solution |
| Hydrastis canadensis | Hydrochlorothiazide | Hydrocodone bitartrate | Hydrocodone polistirex |
| Hydromorphone | Hydroxychloroquine | Hydroxypropyl cellulose | Hyland's calms forté 4 |
| hydrochloride | sulfate | | kids tablets |
| Hyand's calms forté | Hyland's calms forté | Hyland's cold 'n cough 4 | Hyland's colic tablets |
| caplets | tablets | kids | |
| Hyland's earache drops | Hyland's leg cramps PM | Hyland's leg cramps with | Hyland's leg cramps with |
| | with quinine tablets | quinine caplets | quinine tablets |
| Hyland's nerve tonic | Hyland's nerve tonic | Hyland's restful legs | Hyland's sniffles 'n |
| caplets | tablets | tablets | sneezes 4 kids tablets |
| Hyland's teething gel | Hyland's teething tablets | Hyoscine hydrobromide | Hyoscyamine sulfate |
| Hypericum perforatum | Hyzaar 50-12.5 tablets | Hyzaar 100-12.5 tablets | Hyzaar 100-25 tablets |
| Ibandronate sodium | Ibuprofen | Ibuprofen Lysine | Ilaris Injection |
| Iloperidone | Imatinib mesylate | Imipenem | Imiquimod |
| Imitrex injection | Imitrex nasal spray | Imitrex tablets | Immune globulin |
| | | | intravenous (human) |
| Immunizen capsules | Immunocal powder | Imodium A-D liquid | Imodium multi-symptom |
| | sachets | caplets, and EZ chews | relief caplets and |
| | | | chewable tablets |
| Implanon implant | Indapamide | Indinavir sulfate | Indocin capsules |
| Indocin I.V. | Indocin oral suspension | Indocin suppositories | Indomethacin |
| Indomethacin sodium | Infanrix injection vaccine | Infants' strength products | Infliximab |
| trihydrate | | | |
| Influenza virus vaccine | Influenza virus vaccine | Innopran XL extended | Inositol |
| | live, intranasal | release capsules | |
| Insulin, human (RDNA | Insulin aspart, human | Insulin aspart, human | Insulin aspart protamine, |
| origin) | | regular | human |
| Insulin detemir (RDNA | Insulin glargine | Insulin glulisine | Insulin Lispro, human |
| origin) | | | |
| Insulin lispro protamine, | Insulin, human NPH | Insulin, human regular | Insulin, human regular |
| human | | | and human NPH mixture |
| Integra F supplement | Integra plus supplement | Integra supplement | Integrilin injection |
| capsules | capsules | capsules | |
| Interferon alfa-2B, | Interferon alfa-N3 | Interferon beta-1A | Interferon beta-1B |
| recombinant | (human leukocyte | | |
| | derived) | | |
| Interferon gamma-1B | Intravenous sodium diuril | Intron A for injection | Intuniv extended release |
| | | | tablets |
| Invanz for injection | Invega extended-release | Invega sustenna | Iodine |
| | tablets | extended-release | |
| | | injectable suspension | |
| Iodine I 131 tositumomab | Ipratropium bromide | Iquix ophthalmic solution | Irbesartan |
| Iron Carbonyl | Iron Polysaccharide | Isentress Tablets | Isocarboxazid |
| | complex | | |
| Isoflurane | Isotretinoin | Isradipine | Ivermectin |
| Ivy Block | Janumet Tablets | Januvia Tablets | Kaletra Oral Solution |
| Kaletra Tablets | Kapidex Delayed Release | Kepivance | Keppra XR Extended- |
| | Capsules | | Release Tablets |
| Ketek Tablets | Ketoconazole | Ketoprofen | Ketorolac Tromethamine |
| Ketotifen Fumarate | Kineret Injection | Kinrix Injection Vaccine | Klonopin Tablets |
| Klonopin Wafers | Klor-Con s/Klor-Con 10 | Klor-Con M20/Flor-Con | K-Phos Original (Sodium |
| | Tablets | M10/Klor-Con M15 | Free) Tablets |
| | | Tablets | |
| K-Phos M.F. Tablets | K-Phos Neutral Tablets | K-Phos No. 2 Tablets | Kristalose for Oral |
| | | | Solution |
| Lacosamide | Lacrisert Sterile | Lactic Acid | Lactulose |
| | Ophthalmic Insert | | |
| Lamictal Chewable | Lamictal ODT Orally | Lamictal Tablets | Lamictal XR Extended- |
| Dispersible Tablets | Disintegrating Tablets | | Release Tablets |
| Laminaria Hyperborea | Lamivudine | Lamotrigine | Lanoxin Injection |
| Lanoxin Injection | Lanoxin Tablets | Lanthanum Carbonate | Lantus Injection |
| Pediatric | | | |
| Lapatine | L-Arginine | L-Carnitine | L-Cysteine |
| Lepirudin | Letairis Tablets | Letrozole | Leukeran Tablets |
| Leuprolide Acetate | Leustatin Injection | Levaquin Injection | Levaquin Oral Solution |
| Levaquin Tablets | Levaquin in 5% Dextrose | Levemir Injection | Levetiracetam |
| | Injection | | |
| Levitra Tablets | Levitra Tablets (see | Levocarnitine | Levocetirizine |
| | Schering) | | Dihydrochloride |
| Levodopa | Levofloxacin | Levonorgestrel | Levothyroxine Sodium |
| Levoxyl Tablets | Lexapro Oral Suspension | Lexapro Tablets | Lexiscan Injection |
| Lexiva Oral Suspension | Lexiva Tablets | Lialda Tablets | Lidocaine |
| Lidoderm Patch | Lifepak Capsules | Linezolid | Liothyronine Sodium |
| Lipitor Tablets | Lipoic Acid | Lisdexamfetamine | Lisinopril |
| | | Dimesylate | |
| Liver, Dessicated | Liver Fractions | Liver Preparations | L-Lysine |
| Loestrin 24 Fe Tablets | Loperamide | Lopinavir | Lorazepam |
| | Hydrochloride | | |
| Losartan Potassium | Loseasonique Tablets | Lovastatin | Lovaza Capsules |
| Lovenox Injection | Loxapine Hydrochloride | L-Proline | Lubiprostone |
| Lucentis Injection | Lumefantrine | Lumigan Ophthalmic | Lupron Depot 3.75 MG |
| | | Solution | |
| Lupron Depot 7.5 MG | Lupron Depot-3 month | Lupron Depot-3 month | Lupron Depot-4 month |
| | 11.25 MG | 22.5 MG | 30 MG |
| Lupron Depot-Ped 7.5 MG, | Lutein | Lutropin Alfa | Luveris for Injection |
| 11.25 MG and 15 MG | | | |
| Lybrel Tablets | Lycium Barbarum | Lycopodium Clavatum | Lyrica Capsules |
| Mafenide Acetate | Mag-Al Liquid | Mag-Al Plus | Mag-Al Plus XS |
| Mag-Al Ultimate | Magnesium | Magnesium Carbonate | Magnesium Citrate |
| Strength | | | |
| Magnesium Hydroxide | Magnesium Oxide | Magnesium Sulfate | Malarone Pediatric |
| | | | Tablets |
| Malarone Tablets | Manganese | Manganese Sulfate | Maprotiline |
| | | | Hydrochloride |
| Maraviroc | Marineomega Softgel | Maritime Pine Extract | Marplan Tablets |
| | Capsules | | |
| Mavik Tablets | Maxair Autohaler | Maxalt Tablets | Maxalt-MLT Orally |
| | | | Disintegrating Tablets |
| Maximum Strength | Maxzide Tablets | Maxzide-25 MG Tablets | Measles, Mumps, |
| Products | | | Rubella and Varicella |
| | | | Virus Vaccine, Live |
| Measles, Mumps & | Measles Virus Vaccine, | Mechlorethamine | Meclofenamate Sodium |
| Rubella Virus Vaccine, | Live | Hydrochloride | |
| Live | | | |
| Med Omega Fish Oil | Medizym Tablets | Medroxyprogesterone | Mega Antioxidant |
| | | Acetate | Tablets |
| Megace Es Oral | Megestrol Acetate | Meili Soft Capsules | Meili Clear Soft Capsules |
| Suspension | | | |
| Melatonin | Meloxicam | Melphalan | Melphalan Hydrochloride |
| Memantine | Menthol | Mephyton Tablets | Mepron Suspension |
| Hydrochloride | | | |
| Mercaptopurine | Meribin Capsules | Meridia Capsules | Meropenem |
| Merrem I.V. | Meruvax II | Mesalamine | Metadate CD Capsules |
| Metaxalone | Metformin Hydrochloride | Methadone Hydrochloride | Methenamine Mandelate |
| Methionne | Methotrexate Sodium | Methyclothiazide | Methyl Salicylate |
| Methyldopa | Methylnaltrexone | Methylphenidate | Methylphenidate |
| | Bromide | | Hyrdochloride |
| Metoclopramide | Metolazone | Metoprolol Succinate | Metoprolol Tartrate |
| Hydrochloride | | | |
| Metozolov Tablets | Metronidazole | Metyrosine | Mevacor Tablets |
| Micafungin Sodium | Micardis Tablets | Micardis HCT Tablets | Miconazole Nitrate |
| Midodrine Hydrochloride | Milk of Magnesia | Milk of Magnesia | Milk of Magnesia |
| | | Suspension | Concentrate (24% |
| | | | Suspension) |
| Milnacipran | Mineral Oil | Minerals | Minerals, Multiple |
| Hydrochloride | | | |
| Minocycline | Mirtazapine | Mitoxantrone | M-M-R II |
| Hydrochloride | | Hydrochloride | |
| Moban Tablets | Modafinil | Modicon Tablets | Molindone |
| | | | Hydrochloride |
| Molybdenum | Mometasone Furoate | Mometasone Furoate | Monobasic Sodium |
| | | Monohydrate | Phosphate |
| Montelukast Sodium | Morphine Sulfate | Motrin IB Tablets and | Children's Motrin Dosing |
| | | Caplets | Chart |
| Children's Motrin Oral | Children's Motrin Non- | Infants' Motrin | Infants' Motrin Non- |
| Suspension | Staining Dye-Free Oral | Concentrated Drops | Staining Dye-Free |
| | Suspension | | Concentrated Drops |
| Junior Strength Motrin | Moviprep Oral Solution | Moxatag Tablets | Moxifloxacin |
| Caplets and Chewable | | | Hydrochloride |
| Tablets | | | |
| MS Contin Tablets | Multaq Tablets | Multiminerals | Multivitamins |
| Multivitamins with | Mumps Virus Vaccine, | Mumpsvax | Mupirocin |
| Minerals | Live | | |
| Mupirocin Calcium | Muromonab-CD3 | Mustargen for Injection | Mycamine for Injection |
| Mycophenolate Mofetil | Mycophenolic Acid | Myfortic Tablets | Myleran Tablets |
| Mylotarg for Injection | Nadolol | Naftifine Hydrochloride | Nameda Oral Solution |
| Nameda Tablets | Naprosyn Suspension | Naprosyn Tablets | Naproxen |
| Naproxen Sodium | Naratriptan | Nasacort AQ Nasal Spray | Nascobal Nasal Spray |
| | Hydrochloride | | |
| Nasonex Nasal Spray | Natrecor for Injection | Naturethroid Tablets | Nebivolol |
| Nelarabine | Nembutal Sodium | Neoprofen Injection | Neoral Oral Solution |
| | Solution, USP | | |
| Neoral Soft Gelatin | Neulasta Injection | Neupogen for Injection | Nevirapine |
| Capsules | | | |
| Nexium Delayed-Release | Nexium Delayed-Release | Nexium I.V. | Niacin |
| Capsule | Oral Suspension | | |
| Niacinamide | Niaspan Extended- | Nicardipine | Nicotinic Acid |
| | Release Tablets | Hydrochloride | |
| Nifedipine | Nilotnib | Nimbex Injection | Nisoldipine |
| Nitrofurantoin | Nitrofurantoin | Nitroglycerin | Nitrolingual Pumpspray |
| Macrocrystals | Monohydrate | | |
| Nizatidine | Norditropin Cartridges | Norel SR Tablets | Norelgestromin |
| Norethindrone | Norethindrone Acetate | Norflex Injectable | Norfloxacin |
| Norgestimate | Noroxin Tablets | Nortriptyline | Norvir Oral Solution |
| | | Hydrochloride | |
| Norvir Soft Gelatin | Norwegian Cod Liver Oil | Novantrone for Injection | Novolog Injection |
| Capsules | | Concentrate | |
| Novolog Mix 70/30 | Novoseven RT | Noxafil Oral Suspension | Nplate |
| Nucynta Tablets | Nu-Iron 150 Capsules | Nu-Iron Elixir | Nutropin for Injection |
| Nutropin AQ Injection | Nutropin AQ Nuspin | Nutropin AQ Pen | Nuvaring |
| | Injection | Cartridge | |
| Nuvigil Tablets | Nystatin | Octocrylene | Octreotide Acetate |
| Oforta Tablets | Olanzapine | Olive Oil | Olmesartan Medoxomil |
| Olopatadine | Omalizumab | Omega-3-Acid Ethyl | Omega-3 Acids |
| Hydrochloride | | Esters | |
| Omega-3 Polyunsaturates | Omegalife-3 | Omerprazole | Omnicef Capsules |
| | Supplementation | | |
| Omnicef for Oral | Onabotulinumtoxina | Oncaspar Injection | Ondansetron |
| Suspension | | | |
| Ondansetron | Onglyza Tablets | Onion | Onsolis Film |
| Hydrochloride | | | |
| Ontak Vials | Opana Tablets | Opana ER Tablets | Oramorph SR Tablets |
| Orlistat | Orphenadrine | Ortho-Cept Tablets | Ortho Micronor Tablets |
| | Hydrochloride | | |
| Ortho-Novum Tablets | Ortho-Novum 1/50 | Ortho Tri-Cyclen LO | orthoclone OKT3 Sterile |
| | Tablets | Tablets | Solution |
| Ortho-Cyclen Tablets | Oseltamivir Phosphate | Osmoprep Tablets | Ovcon 35 Tablets |
| Ovcon 50 Tablets | Ovidrel Prefilled Syringe | Oxaliplatin | Oxybenzone |
| | For Injection | | |
| Oxybutynin Chloride | Oxycodone | Oxycontin Tablets | Oxymetazoline |
| | Hydrochloride | | Hydrochloride |
| Oxymorphone | Palifermin | Paliperidone | Palivizumab |
| Hydrochloride | | | |
| Palonosetron | Pancreatin | Pancrelipase | Panhematin For Injection |
| Hydrochloride | | | |
| Panitumumab | Pantoprazole Sodium | Pantothenate, Calcium | Pantothenic Acid |
| Papain | Parafon Forte DSC | Paricalcitol | Parnate Tablets |
| Paroxetine | Paroxetine Hydrochloride | Paser Granules | Pataday Ophthalmic |
| | | | Solution |
| Patanase Nasal Spray | Paxil Oral Suspension | Paxil Tablets | Paxil CR Controlled- |
| | | | Release Tablets |
| Pediarix Vaccine | Liquid Pedvaxhib | PEG-3350 | Pegasparagase |
| Pegfilgrastim | Peginterferon Alfa-2B | Pegintron Powder For | Pemetrexed Disodium |
| | | Injection | |
| Pemirolast Potassium | Penciclovir | PenicillaminePenicillin G | Penicillin G Procaine |
| | | Benzathine | |
| Pentasa Capsules | Pentobarbital Sodium | Pentosan Polysulfate | Pentoxifylline |
| | | Sodium | |
| Pepcid Tablets | Maximum Strength | Percocet Tablets | Percodan Tablets |
| | Pepcid AC Tablets | | |
| Perforomist Inhalation | Permethrin | Perphenazine | Petrolatum, White |
| Solution | | | |
| Phenazopyridine | Phenobarbital | Phenol | Phenoxybenzamine |
| Hydrochloride | | | Hydrochloride |
| Phentermine | Phenylazodiamino | Phenylephrine | Phenyltoloxamine Citrate |
| Hydrochloride | Pyridine Hydrochloride | Hydrochloride | |
| Phenytek Capsules | Phenytoin Sodium | Extended Phenytoin | Phosphorus |
| | | Sodium Capsules | |
| Photofrin For Injection | Phytonadione | Phytosterols | Pilocarpine |
| | | | Hydrochloride |
| Pimecrolimus | Pindolol | Pink Bismuth | Pioglitazone |
| | | | Hydrochloride |
| Piperacillin Sodium | Pirbuterol Acetate | Piroxicam | Pitcher Plant Distillate |
| Plan B One-Step Tablets | Plasma/Albumin-Free | Plavix Tablets | Pneumococcal Vaccine, |
| | | | Diphtheria Conjugate |
| Pneumococcal Vaccine, | Pneumovax 23 | Policosanol | Polifeprosan 20 With |
| Polyvalent | | | Carmustine |
| Poliovirus Vaccine | Polyethylene Glycol | Polysaccharide Iron | Porfimer Sodium |
| Inactivated | | Complex | |
| Posaconazole | Potaba Capsules | Potaba Tablets | Potassium |
| Potassium Acid | Potassium Chloride | Potassium Citrate | Potassium Iodide |
| Phosphate | | | |
| Potassium Phosphate | Pramlintide Acetate | Prasugrel Hydrochloride | Pravastatin Sodium |
| Prazosin Hydrochloride | Prednisolone Sodium | Pregabalin | Premarin Intraveous |
| | Phosphate | | |
| Premarin Tablets | Premphase Tablets | Prempro Tablets | Prenexa Capsules |
| Prevnar | Primaxin I.M. | Primaxin I.V. | Prinivil Tablets |
| Prinzide Tablets | Pristiq Extended-Release | Proair HFA Inhalation | Probenecid |
| | Tablets | Aerosol | |
| Prochlorperazine Maleate | Procosa II Tablets | Procrit For Injection | Profilnine SD |
| Proflavanol 90 Tablets | Progesterone | Proglycem Capsules | Proglycem Suspension |
| Prograf Capsules | Prograf Injection | Proguanil Hydrochloride | Prolastin |
| Proleukin For Injection | Promacta Tablets | Promethazine | Prometrium Capsules |
| | | Hydrochloride | (100 MG, 200 MG) |
| Propafenone | Propecia Tablets | Propoxyphene | Propoxyphene Napsylate |
| Hydrochloride | | Hydrochloride | |
| Propranolol | Propylene Glycol | Proquad | Proscar Tablets |
| Hydrochloride | | | |
| Proteolytic Enzymes | Protonix For Delayed- | Protonix Delayed-Release | Protonix |
| | Release Oral Suspension | Tablets | |
| Protopic Ointment | Proventil HFA Inhalation | Provigil Tablets | Prozac Weekly Capsules |
| | Aerosol | | |
| Prozac Pulvules | Pseudoephedrine | Pseudoephedrine Sulfate | Pulmicort Flexhaler |
| | Hydrochloride | | |
| Pulmozyme Inhalation | Pulsatilla Pratensis | Pylera Capsules | Pyridium Tablets |
| Solution | | | |
| Pyrimethamine | Quadrivalent Human | Quetiapine Fumarate | Quinapril Hydrochloride |
| | Papillomavirum (Types | | |
| | 6, 11, 16, 18) | | |
| | Recombinant Vaccine | | |
| Quinine | Quixin Ophthalmic | Qvar Inhalation Aerosol | Raberprazole Sodium |
| Raloxifene | Raltegravir | Ramelteon | Ranexa Extended- |
| Hydrochloride | | | Release Tablets |
| Ranibizumab | Ranitidine Hydrochloride | Ranolazine | Rapamune Oral Solution |
| Rapamune Tablets | Rasagiline Mesylate | Rasburicase | Razadyne Oral Solution |
| Razadyne Tablets | Razadyne ER Extended- | Rebetol Capsules | Rebetol Oral Solution |
| | Release Capsules | | |
| Rebif Prefilled Syringe | Reclast Injection | Recombinate | Recombivax HB |
| For Injection | | | |
| Refacto Vials | Refludan For Injection | Regadenoson | Regular Strength |
| | | | Products |
| Reishimax GLP Capsules | Relenza Inhalation | Relistor Injection | Relistor Injection |
| | Powder | | |
| Remeron Tablets | Remeronsoltab Tablets | Remicade For IV | Renacidin Irrigation |
| | | Injection | |
| Reopro Vials | Requip Tablets | Requip XL Tablets | Restasis Ophthalmic |
| | | | Emulsion |
| Retapamulin | Retrovir Capsules | Retrovir IV Infusion | Retrovir Syrup |
| Retrovir Tablets | RH₉ (D) Immune | Rhus Toxicodendron | Ribavirin |
| | Globulin (Human) | | |
| Ribes Nigrum | Riboflavin | Rifaximin | Rilonacept |
| Rilutek Tablets | Riluzole | Risedronate Sodium | Risperdal M-Tab |
| Risperdal Oral Solution | Risperdal Tablets | Risperdal Consta Long- | Risperidone |
| | | Acting Injection | |
| Ritonavir | Rituxan | Rituximab | Rivastigmine Tartrate |
| Rizathiptan Benzoate | Rocephin Injectable Vials | Rocuronium Bromide | Extra Strength Rolaids |
| | | | Softchews Vanilla Creme |
| Romazicon Injection | Romiplostim | Ropinirole Hydrochloride | Rosiglitazone Maleate |
| Rosuvastatin Calcium | Rotarix Oral Suspension | Rotateq | Rotavirus Vaccine, Live, |
| | | | Oral |
| Rotavirus Vaccine, Live, | Roxanol Oral Solution | Roxicodone Oral Solution | Roxicodone Tablets |
| Oral, Pentavalent | | | |
| Rozerem Tablets | Rubella Virus Vaccine, | Rufinamide | Rythmol Tablets |
| | Live | | |
| Rythmol SR Extended | Ryzolt Extended-Release | Sabril Oral Solution | Sabril Tablets |
| Release Capsules | Tablets | | |
| St. Joseph 81 MG Aspirin | Saizen For Injection | Salagen Tablets | Salmeterol Xinafoate |
| Chewable and Enteric | | | |
| Coated Tablets | | | |
| Salmon Oil | Salonpas Arthritis | Salonpas Pain Relief | Sandostatin Injection |
| | | Patch | |
| Sandostatin LAR Depot | Santyl Collagenase | Saphris Tablets | Sarafem |
| | Ointment | | |
| Sarapin Vials | Sarraceniaceae | Savella Tablets | Saxagliptin |
| Scopolamine | Scopolamine | Seasonique Tablets | Selegiline |
| | Hydrobromide | | |
| Selegiline Hydrochloride | Selenium | Selzentry Tablets | Senna |
| Sennosides | Sen-Sei-Ro Powder Gold | Sensipar Tablets | Serevent Diskus |
| Seromycin Capsules | Seroquel Tablets | Seroquel XR Extended- | Serostim For Injection |
| | | Release Tablets | |
| Sertraline Hydrochloride | Sevoflurane | Sheep Placenta | Sibutramine |
| | | | Hydrochloride |
| | | | Monohydrate |
| Silicea | Silicone | Simcor Tablets | Simethicone |
| Simponi Injection | Simulect For Injection | Simvastatin | Singulair Tablets |
| Singular Oral Granules | Sirolimus | Sitagliptin Phosphate | Skelaxin Tablets |
| Slo-Niacin Tablets | Sodium | Sodium Acid Phosphate | Sodium Ascorbate |
| Sodium Chloride | Sodium Citrate | Sodium Fluoride | Sodium Hyaluronate |
| Sodium Oxychlorosene | Sodium Phosphate | Sodium Sulfacetamide | Sodium Sulfate |
| Solifenacin Succinate | Soliris Concentrated | Solodyn Extended | Somatostatin Analogue |
| | Solution for Intravenous | Release Tablets | |
| | Infusion | | |
| Somatropin | Somatropin (RDNA | Son Formula Tablets | Sorbitol |
| | Origin) | | |
| Sore Throat Spray | Soriatane Capsules | Sotalol Hydrochloride | Soy Oil |
| Spacer, Inhalation | Spiriva Handihaler | Spironolactone | Spirulina |
| Springcode Spray | Stalevo Tablets | Stavudine | Strattera Capsules |
| Striant Mucoadhesive | Stromectol Tablets | Succimer | Sucralfate |
| Sudafed 12 Hour Nasal | Sudafed 24 Hour Non- | Sudafed Nasal | Sudafed PE Nasal |
| Decongestant Non- | Drowsy Nasal | Decongestant Tablets | Decongestant Tablets |
| Drowsy Caplets | Decongestant Tablets | | |
| Children's Sudafed Nasal | Children's Sudafed PE | Sudafed OM Sinus | Sulfamethoxazole |
| Decongestant Liquid | Nasal Decongestant | Congestion Moisturizing | |
| | Liquid | Nasal Spray | |
| Sulfur | Sulindac | Sumatriptan | Sumatriptan Succinate |
| Sunitinib Malate | Super Omega-3 Softgels | Supprelin La Implant | Suprane Liquid for |
| | | | Inhalation |
| Suprax for Oral | Suprax Tablets | Sutent Capsules | Symbicort 80/4.5 |
| Suspension | | | Inhalation Aerosol |
| Symbicort 160/4.5 | Symbyax Capsules | Symlin Injection | Symlinpen |
| Inhalation Aerosol | | | |
| Symphytum Officinale | Synagis Intramuscular | Synthroid Tablets | Syprine Capsules |
| | Solution | | |
| Systane Ultra Lubricant | Tabloid Tablets | Taclonex Ointment | Taclonex Scalp Topical |
| Eye Drops | | | Suspension |
| Tacrolimus | Tadalafil | Tambocor Tablets | Tamiflu Capsules |
| Tamiflu Oral Suspension | Tamoxifen Citrate | Tandem Capsules | Tandem DHA Capsules |
| Tandem F. Capsules | Tandem OB Capsules | Tandem Plus Capsules | Tapentadol |
| | | | Hydrochloride |
| Tarceva Tablets | Targretin Capsules | Tarka Tablets | Tasigna Capsules |
| Taurine | Taxotere Injection | Tazobactam Sodium | Tegreen 97 Capsules |
| | Concentrate | | |
| Tekturna Tablets | Tekturna HCT Tablets | Telithromycin | Telmesteine |
| Telmisartan | Temazepam | Temodar Capsules | Temodar Injection |
| Temozolomide | Temsirolimus | Tenecteplase | Tenofovir Disoproxil |
| | | | Fumarate |
| Terazol 3 Vaginal | Terazosin Hydrochloride | Terbinafine | Teriparatide |
| Suppositories | | Hydrochloride | |
| Testosterone | Testosterone Enanthate | Tetrabenazine | Tetracycline |
| | | | Hydrochloride |
| Teveten Tablets | Teveten HCT Tablets | Tev-Tropin for Injection | Theophylline |
| Theophylline Anhydrous | Thiamine Disulfide | Thiamine Mononitrate | Thioguanine |
| Thioridazine | Thiothixene | Thymus Polypeptide | Thyroid |
| Hydrochloride | | | |
| Tiagabine Hydrochloride | Ticarcillin Disodium | Tice BCG | Tigecycline |
| Timentin Add-Vantage | Timentin Injection | Timentin IV Infusion | Timentin Pharmacy Bulk |
| | Galaxy Container | | Package |
| Timolol Hemihydrate | Timolol Maleate | Timoptic In Ocudose | Timoptic Sterile |
| | | | Ophthalmic Solution |
| Tiotropium Bromide | Tizanidine | Tnkase | Tobi Nebulizer Solution |
| | | | for Inhalation |
| Tobramycin | Tocopheryl Acetate | Tolazamide | Tolbutamide |
| Tolectin 200/400/600 | Tolmetin Sodium | Topamax Sprinkle | Topamax Tablets |
| | | Capsules | |
| Topiramate | Topotecan Hydrochloride | Toprol-XL Tablets | Torisel Injection |
| Tositumomab | Toviaz Extended-Release | Tracleer Tablets | Tramadol Hydrochloride |
| | Tablets | | |
| Trandolapril | Tranylcypromine Sulfate | Trastuzumab | Traumeel Ear Drops |
| Traumeel Injection | Traumeel Oral Drops | Traumeel Oral Liquid In | Traumeel Tablets |
| Solution | | Vials | |
| Travatan Z Ophthalmic | Travoprost | Treanda For Injection | Tretinoin |
| Solution | | | |
| Treximet Tablets | Triamcinolone Acetonide | Triamterene | Tribasic Calcium |
| | | | Phosphate |
| Tricitrates Oral Solution | Tricitrates SF Oral | Tricor Tablets | Trientine Hydrochloride |
| | Solution | | |
| Trifluoperazine | Trihexyphenidyl | Trilipix Delayed Release | Trimethoprim |
| Hydrochloride | Hydrochloride | Capsules | |
| Trisenox Injection | Trizivir Tablets | Trusopt Sterile | Truvada Tablets |
| | | Ophthalmic Solution | |
| Trypsin | Tussionex Pennkinetic | Twinject Auto-Injector | Tygacil for Injection |
| | Extended-Release | | |
| | Suspension | | |
| Tykerb Tablets | Regular Strength Tylenol | Tylox Capsules | Uloric Tablets |
| | Tables | | |
| Ultane Liquid For | Ultracet Tablets | Ultram Tablets | Ultram ER Extended- |
| Inhalation | | | Release Tablets |
| Ultrase Capsules | Ultrase MT Capsules | Undecylenic Acid | Uniphyl Tablets |
| Urocit-K Tablets | Uroqid-Acid No. 2 | Uroxatral Tablets | Urso 250 Tablets |
| | Tablets | | |
| Urso Forte Tablets | Ursodiol | Vagifem Tablets | Valacyclovir |
| | | | Hydrochloride |
| Valcyte Tablets | Valcyte For Oral Solution | Valganciclovir | Valium Tablets |
| | | Hydrochloride | |
| Valproic Acid | Valrubicin | Valsartan | Valstar Sterile Solution |
| | | | For Intravesical |
| | | | Instillation |
| Valtrex Caplets | Valturna Tablets | Vanadium | Vantas Implant |
| Vaprisol | Vaqta | Vardenafil Hydrochloride | Varenicline Tartrate |
| Varicella Virus Vaccine, | Varivax | Vectribix | Velcade For Injection |
| Live | | | |
| Venlafaxine | Ventolin HFA Inhalation | Veramyst Nasal Spray | Verapamil Hydrochloride |
| Hydrochloride | Aerosol | | |
| Verteporfin | Vesicare Tablets | Vicodin Tablets | Vicodin ES Tablets |
| Vicodin HP Tablets | Vicoprofen Tablets | Vigabatrin | Vigamox Ophthalmic |
| | | | Solution |
| Vimpat Injection | Vimpat Tablets | Viokase Powder | Viokase Tablets |
| Viramune Oral | Viramune Tablets | Viread Tablets | Visudyne For Injection |
| Suspension | | | |
| Visutein Capsules | Vitamin A | Vitamin B1 | Vitamin B2 |
| Vitamin B6 | Vitamin B12 | Vitamin C | Vitamin D |
| Vitamin D3 | Vitamin E | Vitamin K | Vitamin K1 |
| Vitamins, Multiple | Vitamins, Prenatal | Vitamins with Minerals | Vitis Vinifera |
| Von Willebrand Factor | Vorinostat | Vytorin 10/10 Tablets | Vytorin 10/10 Tablets |
| (Human) | | | |
| Vytorin 10/20 Tablets | Vytorin 10/40 Tablets | Vytorin 10/80 Tablets | Vyvanse Capsules |
| Watchhaler | Welchol Tablets | Wellburtrin Tablets | Wellbutrin SR Sustained- |
| | | | Release Tablets |
| Westhroid Tablets | White Petrolatum | Winrho SDF | Xeloda Tablets |
| Xenazine Tablets | Xenical Capsules | Xifaxan Tablets | Xigris Powder For |
| | | | Intravenous Infusion |
| Xolair | Xolair | Xyntha Vials | Xyzal Oral Solution |
| Xyzal Oral Solution | Xyzal Tablets | Xyzal Tablets | Yarrow |
| Yaz Tablets | Yeast | Zafirlukast | Zaleplon |
| Zanamivir | Zantac 25 Efferdose | Zantac 150 Tablets | Zantac 300 Tablets |
| | Tablets | | |
| Zantac Injection | Zantac Injection | Zantac Injection | Zantac Syrup |
| | Pharmacy Bulk Package | Premixed | |
| Zeaxanthin | Zeel Injection Solution | Zemplar Capsules | Zemplar Injection |
| Zemuron Injection | Zetia Tablets | Zetia Tablets | Ziagen Oral Solution |
| Ziagen Tablets | Zidovudine | Zinacef For Injection | Zinacef Injection |
| Zinc | Zinc Citrate | Zinc Oxide | Zinc Sulfate |
| Zinc-220 Capsules | Ziprasidone | Ziprasidone Mesylate | Zipsor 25 MG Liquid |
| | Hydrochloride | | Filled Capsules |
| Zocor Tablets | Zofran Injection | Zofran Injection | Zofran Oral Solution |
| | | Premixed | |
| Zofran Tablets | Zofran ODT Orally | Zoledronic Acid | Zolinza Capsules |
| | Disintegrating Tablets | | |
| Zolmitriptan | Zolpidem Tartrate | Zometa For Intravenous | Zomig Tablets |
| | | Infusion | |
| Zomig Nasal Spray | Zomig-ZMT Tablets | Zonegran Capsules | Zonisamide |
| Zorbtive For Injection | Zostavax Injection | Zoster Vaccine Live | Zosyn for Injection |
| Zovirax Capsules | Zovirax Suspension | Zovirax Tablets | Zyban Sustained-Release |
| | | | Tablets |
| Zydone Tablets | Zyprexa Tablets | Zyprexa Intramuscular | Zyprexa Zydis Orally |
| | | | Disintegrating Tablets |
| Zyrtec Allergy Tablets | Zyvox For Oral | Zyvox Injection | Zyvox Tablets |
| | Suspension | | |

### Nanoparticles

Nanoparticles are provided which are functionalized to have an oligonucleotide attached thereto. The size, shape and chemical composition of the nanoparticles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. Mixtures of nanoparticles having different sizes, shapes and/or chemical compositions, as well as the use of nanoparticles having uniform sizes, shapes and chemical composition, and therefore a mixture of properties are contemplated. Examples of suitable particles include, without limitation, aggregate particles, isotropic (such as spherical particles), anisotropic particles (such as non-spherical rods, tetrahedral, and/or prisms) and core-shell particles, such as those described in U.S. Pat. No. 7,238,472 and International Publication No. WO 2003/08539, the disclosures of which are incorporated by reference in their entirety.

In one embodiment, the nanoparticle is metallic, and in various aspects, the nanoparticle is a colloidal metal. Thus, in various embodiments, nanoparticles of the invention include metal (including for example and without limitation, silver, gold, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials.

Also, as described in U.S. Patent Publication No 2003/0147966, nanoparticles of the invention include those that are available commercially, as well as those that are synthesized, e.g., produced from progressive nucleation in solution (e.g., by colloid reaction) or by various physical and chemical vapor deposition processes, such as sputter deposition. See, e.g., HaVashi, Vac. Sci. Technol. A5(4):1375-84 (1987); Hayashi, Physics Today, 44-60 (1987); MRS Bulletin, January 1990, 16-47. As further described in U.S. Patent Publication No 2003/0147966, nanoparticles contemplated are alternatively produced using HAuCl₄ and a citrate-reducing agent, using methods known in the art. See, e.g., Marinakos et al., Adv. Mater. 11:34-37 (1999); Marinakos et al., Chem. Mater. 10: 1214-19 (1998); Enustun & Turkevich, J. Am. Chem. Soc. 85: 3317 (1963).

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein.

### Oligonucleotides

Oligonucleotides contemplated by the present disclosure include DNA, RNA and modified forms thereof as defined herein. An “oligonucleotide” is understood in the art to comprise individually polymerized nucleotide subunits. The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases adenine (A), guanine (G), cytosine (C), thymine (T) and uracil (U). Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleotides include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine(1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the oligonucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with an oligonucleotide, or a modified form thereof, and optionally a domain as defined herein below, generally comprise an oligonucleotide from about 5 nucleotides to about 100 nucleotides in length. More specifically, nanoparticles are functionalized with oligonucleotides that are about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all oligonucleotides intermediate in length of the sizes specifically disclosed to the extent that the oligonucleotide is able to achieve the desired result. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length are contemplated.

In some aspects, nanoparticles with an oligonucleotide and a therapeutic agent attached thereto are provided wherein an oligonucleotide further comprising a domain is associated with the nanoparticle. The domain that is part of the oligonucleotide-functionalized nanoparticle as described herein affects the efficiency with which the nanoparticle is taken up by a cell. Accordingly, the domain increases or decreases the efficiency. As used herein, “efficiency” refers to the number or rate of uptake of nanoparticles in/by a cell. Because the process of nanoparticles entering and exiting a cell is a dynamic one, efficiency can be increased by taking up more nanoparticles or by retaining those nanoparticles that enter the cell for a longer period of time. Similarly, efficiency can be decreased by taking up fewer nanoparticles or by retaining those nanoparticles that enter the cell for a shorter period of time.

The domain, in some aspects, is contiguous/colinear with the oligonucleotide and is located proximally with respect to a nanoparticle. In some aspects, the domain is contiguous/colinear with the oligonucleotide and is located distally with respect to a nanoparticle. The terms “proximal” and “distal” refer to a position relative to the midpoint of the oligonucleotide. In some aspects, the domain is located at an internal region within the oligonucleotide. In further aspects, the domain is located on a second oligonucleotide that is attached to a nanoparticle. Accordingly, a domain, in some embodiments, is contemplated to be attached to a nanoparticle as a separate entity from an oligonucleotide.

It is further contemplated that an oligonucleotide, in some embodiments, comprise more than one domain, located at any of the locations described herein.

The domain, in some embodiments, increases the efficiency of uptake of the oligonucleotide-functionalized nanoparticle by a cell. In some aspects, the domain comprises a sequence of thymidine residues (polyT) or uridine residues (polyU). In further aspects, the polyT or polyU sequence comprises two thymidines or uridines. In various aspects, the polyT or polyU sequence comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 125, about 150, about 175, about 200, about 250, about 300, about 350, about 400, about 450, about 500 or more thymidine or uridine residues.

In some embodiments, it is contemplated that a nanoparticle functionalized with an oligonucleotide, a therapeutic agent and a domain is taken up by a cell with greater efficiency than a nanoparticle functionalized with the same oligonucleotide but lacking the domain. In some aspects, a nanoparticle functionalized with an oligonucleotide, a therapeutic agent and a domain is taken up by a cell 1% more efficiently than a nanoparticle functionalized with the same oligonucleotide but lacking the domain. In various aspects, a nanoparticle functionalized with an oligonucleotide, a therapeutic agent and a domain is taken up by a cell 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 100-fold or higher, more efficiently than a nanoparticle functionalized with the same oligonucleotide and therapeutic agent but lacking the domain.

In some embodiments, the domain decreases the efficiency of uptake of the oligonucleotide-functionalized nanoparticle by a cell. In some aspects, the domain comprises a phosphate polymer (C3 residue) that is comprised of two phosphates. In various aspects, the C3 residue comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 125, about 150, about 175, about 200, about 250, about 300, about 350, about 400, about 450, about 500 or more phosphates.

In some embodiments, it is contemplated that a nanoparticle functionalized with an oligonucleotide, a therapeutic agent and a domain is taken up by a cell with lower efficiency than a nanoparticle functionalized with the same oligonucleotide but lacking the domain. In some aspects, a nanoparticle functionalized with an oligonucleotide, a therapeutic agent and a domain is taken up by a cell 1% less efficiently than a nanoparticle functionalized with the same oligonucleotide but lacking the domain. In various aspects, a nanoparticle functionalized with an oligonucleotide and a domain is taken up by a cell 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 100-fold or higher, less efficiently than a nanoparticle functionalized with the same oligonucleotide and therapeutic agent but lacking the domain.

### Attachment of a Therapeutic Agent

The disclosure provides, in some embodiments, ON-NPs wherein a therapeutic agent is attached to the oligonucleotide. Methods of attaching a therapeutic agent or a chemotherapeutic agent to an oligonucleotide are known in the art, and are described in Priest, U.S. Pat. No. 5,391,723, Arnold, Jr., et al., U.S. Pat. No. 5,585,481, Reed et al., U.S. Pat. No. 5,512,667 and PCT/US2006/022325, the disclosures of which are incorporated herein by reference in their entirety).

### Modified Oligonucleotides

As discussed above, modified oligonucleotides are contemplated for functionalizing nanoparticles. In various aspects, an oligonucleotide functionalized on a nanoparticle is completely modified or partially modified. Thus, in various aspects, one or more, or all, sugar and/or one or more or all internucleotide linkages of the nucleotide units in the oligonucleotide are replaced with “non-naturally occurring” groups.

In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed oligonucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et. al., Current Opinion in Structural Biology 5:343-355 (1995) and Susan M. Freier and Karl-Heinz Altmann, Nucleic Acids Research, 25:4429-4443 (1997), the disclosures of which are incorporated herein by reference.

Specific examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide.”

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated.

Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In still other embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In various forms, the linkage between two successive monomers in the oligonucleotide consists of 2 to 4, desirably 3, groups/atoms selected from —CH₂—, —O—, —S—, —NRH—, >C═O, >C═NRH, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHRH)—, where RH is selected from hydrogen and C1-4-alkyl, and R″ is selected from C1-6-alkyl and phenyl. Illustrative examples of such linkages are CH₂—CH₂—CH₂—, —CH₂—CO—CH₂—, —CH₂—CHOH—CH₂—, —O—CH2-O—, —O—CH2-CH2-, —O—CH2-CH=(including R5 when used as a linkage to a succeeding monomer), —CH₂—CH₂—O—, —NRH—CH₂—CH₂—, —CH₂—CH₂—NRH—, —CH₂—NRH—CH₂—, —O—CH₂—CH₂—NRH—, —NRH—CO—O—, —NRH—CO—NRH—, —NRH—CS—NRH—, —NRH—C(═NRH)—NRH—, —NRH—CO—CH₂—NRH—O—CO—O—, —O—CO—CH₂—O—, —O—CH₂—CO—O—, —CH₂—CO—NRH—, —O—CO—NRH—, —NRH—CO—CH₂—, O—CH₂—CO—NRH—, —O—CH₂—CH₂—NRH—, —CH═N—O—, —CH₂—NRH—O—, —CH₂—O—N=(including R5 when used as a linkage to a succeeding monomer), —CH₂—O—NRH—, —CO—NRH—CH₂—, —CH₂—NRH—O—, —CH₂—NRH—CO—, —O—NRH—CH₂—, —O—NRH, —O—CH₂—S—, —S—CH₂—O—, —CH₂—CH₂—S—, —O—CH₂—CH₂—S—, —S—CH₂—CH=(including R5 when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NRH—, —NRH—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHRN)—O—, —O—P(O)₂—NRHH—, —NRH—P(O)₂—O—, —O—P(O,NRH)—O—, —CH₂—P(O)₂—O—, —O—P(O)₂—CH₂—, and —O—Si(R″)₂—O—; among which —CH₂—CO—NRH—, —CH₂—NRH—O—, —S—CH₂—O—, —O—P(O)₂—O—O—P(—O,S)—O—, —O—P(S)₂—O—, —NRHP(O)₂—O—, —O—P(O,NRH)—O—, —O—PO(R″)—O—, —O—PO(CH₃)—O—, and—O—PO(NHRN)—O—, where RH is selected form hydrogen and C1-4-alkyl, and R″ is selected from C1-6-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of oligonucleotides are described in detail in U.S. Patent Application No. 20040219565, the disclosure of which is incorporated by reference herein in its entirety.

Modified oligonucleotides may also contain one or more substituted sugar moieties. In certain aspects, oligonucleotides comprise one of the following at the 2° position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH2)ₙOCH₃, O(CH₂)ₙNH₂, O(CH₂)ₙCH₃, O(CH₂)ₙONH₂, and O(CH₂)ₙON[CH₂)ₙCH₃]₂, where n and m are from 1 to about 10. Other oligonucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., 1995, Helv. Chim. Acta, 78: 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′—O—CH₂—O—CH₂—N(CH₃)₂.

Still other modifications include 2′-methoxy (2′—O—CH₃), 2′-aminopropoxy (2′-OCH₂CH₂CH₂NH₂), 2′-allyl (2′—CH₂—CH═CH₂), 2′—O-allyl (2′—O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects a methylene (—CH₂)n group bridging the 2′ oxygen atom and the 4′ carbon atom wherein n is 1 or 2. LNAs and preparation thereof are described in WO 98/39352 and WO 99/14226, the disclosures of which are incorporated herein by reference.

### Oligonucleotide Attachment to a Nanoparticle

Oligonucleotides contemplated for use in the methods include those bound to the nanoparticle through any means. Regardless of the means by which the oligonucleotide is attached to the nanoparticle, attachment in various aspects is effected through a 5′ linkage, a 3′ linkage, some type of internal linkage, or any combination of these attachments.

Functionalized NPs can be prepared with both antisense oligonucleotides and peptides designed to affect intracellular localization. The synthetic strategy, in various aspects, uses thiolated oligonucleotides and cystine-terminated peptides to modify the NP surfaces.

Methods of attachment are known to those of ordinary skill in the art and are described in US Publication No. 2009/0209629, which is incorporated by reference herein in its entirety. Methods of attaching RNA to a nanoparticle are generally described in PCT/US2009/65822, which is incorporated by reference herein in its entirety. Accordingly, in some embodiments, the disclosure contemplates that an oligonucleotide attached to a nanoparticle is RNA.

In some embodiments, the oligonucleotide attached to a nanoparticle is DNA. When DNA is attached to the nanoparticle, the DNA is comprised of a sequence that is sufficiently complementary to a target sequence of an oligonucleotide such that hybridization of the DNA oligonucleotide attached to a nanoparticle and the target oligonucleotide takes place, thereby associating the target oligonucleotide to the nanoparticle. The DNA in various aspects is single stranded or double-stranded, as long as the double-stranded molecule also includes a single strand sequence that hybridizes to a single strand sequence of the target oligonucleotide. In some aspects, hybridization of the oligonucleotide functionalized on the nanoparticle can form a triplex structure with a double-stranded target oligonucleotide. In another aspect, a triplex structure can be formed by hybridization of a double-stranded oligonucleotide functionalized on a nanoparticle to a single-stranded target oligonucleotide.

### Spacers

In certain aspects, functionalized nanoparticles are contemplated which include those wherein an oligonucleotide is attached to the nanoparticle through a spacer. “Spacer” as used herein means a moiety that does not participate in modulating gene expression per se but which serves to increase distance between the nanoparticle and the oligonucleotide, or to increase distance between individual oligonucleotides when attached to the nanoparticle in multiple copies, or to increase distance between the therapeutic agent and the nanoparticle. Thus, spacers are contemplated being located between individual oligonucleotides in tandem, whether the oligonucleotides have the same sequence or have different sequences. In aspects of the invention where a domain is attached directly to a nanoparticle, the domain is optionally functionalized to the nanoparticle through a spacer. In aspects wherein domains in tandem are functionalized to a nanoparticle, spacers are optionally between some or all of the domain units in the tandem structure. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, an ethylglycol, or combinations thereof.

Spacers, in some embodiments, include cleavable linkers. A “cleavable linker” as used herein facilitates release of a therapeutic agent in a cell. For example and without limitation, an acid-labile linker, peptidase-sensitive linker, dimethyl linker or disulfide-containing linker [Chari et al. Cancer Research 52: 127-131 (1992)], esters and hydrazones that are relatively stable at physiological pH, but are labile in the acidic endosomal environment, may be used. Accordingly, therapeutic agents of the present disclosure are, in some aspects, bound to the NP surface via a number of different cleavable linkers designed to release the drug upon entering a cell. Other cleavable linkers include without limitation peptides that are cleaved by cancer-specific enzymes, such as matrix metalloproteases.

In certain aspects, the oligonucleotide has a spacer through which it is covalently bound to the nanoparticles. These oligonucleotides are the same oligonucleotides as described above. In instances wherein the spacer is an oligonucleotide, the length of the spacer in various embodiments is at least about 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, at least 50 nucleotides, or even greater than 50 nucleotides. The spacer may have any sequence which does not interfere with the ability of the oligonucleotides to become bound to the nanoparticles or to facilitate uptake of the functionalized nanoparticle. The spacers should not have sequences complementary to each other or to that of the oligonucleotides. In certain aspects, the bases of the oligonucleotide spacer are all adenines, all thymines, all cytidines, all guanines, all uracils, or all some other modified base.

### Surface Density

The density of oligonucleotides on the surface of the NP can be tuned for a given application. For instance, work by Seferos et al. [Nano Lett., 9(1): 308-311, 2009] demonstrated that the density of DNA on the NP surface affected the rate at which it was degraded by nucleases. This density modification is used, for example, in a NP based therapeutic agent delivery system where a drug and ON-NP enter cells, and the ON is degraded at a controlled rate.

Accordingly, nanoparticles as provided herein have a packing density of the oligonucleotides on the surface of the nanoparticle that is, in various aspects, sufficient to result in cooperative behavior between nanoparticles and between oligonucleotide strands on a single nanoparticle. In another aspect, the cooperative behavior between the nanoparticles increases the resistance of the oligonucleotide to nuclease degradation. In yet another aspect, the uptake of nanoparticles by a cell is influenced by the density of oligonucleotides associated with the nanoparticle. As described in PCT/US2008/65366, incorporated herein by reference in its entirety, a higher density of oligonucleotides on the surface of a nanoparticle is associated with an increased uptake of nanoparticles by a cell.

A surface density adequate to make the nanoparticles stable and the conditions necessary to obtain it for a desired combination of nanoparticles and oligonucleotides can be determined empirically. Generally, a surface density of at least 2 pmoles/cm² will be adequate to provide stable nanoparticle-oligonucleotide compositions. In some aspects, the surface density is at least 15 pmoles/cm². Methods are also provided wherein the oligonucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

### Targeting Moiety

The term “targeting moiety” as used herein refers to any molecular structure which assists a compound or other molecule in binding or otherwise localizing to a particular target, a target area, entering target cell(s), or binding to a target receptor. For example and without limitation, targeting moieties may include proteins, peptides, aptamers, lipids (including cationic, neutral, and steroidal lipids, virosomes, and liposomes), antibodies, lectins, ligands, sugars, steroids, hormones, and nutrients, may serve as targeting moieties.

In some embodiments, the targeting moiety is a protein. The protein portion of the composition of the present disclosure is, in some aspects, a protein capable of targeting the composition to target cell. Such a targeting protein may be a protein, polypeptide, or fragment thereof that is capable of binding to a desired target site in vivo. The targeting protein of the present disclosure may bind to a receptor, substrate, antigenic determinant, or other binding site on a target cell or other target site.

A targeting protein may be modified (for example and without limitation, to produce variants and fragments of the protein), as long as the desired biological property of binding to its target site is retained. A targeting protein may be modified by using various genetic engineering or protein engineering techniques. Typically, a protein will be modified to more efficiently bind to the target cell binding site. Such modifications are known and are routine to one of skill in the art.

Examples of targeting proteins include, but are not limited to, antibodies and antibody fragments; serum proteins; fibrinolytic enzymes; peptide hormones; and biologic response modifiers. Among the suitable biologic response modifiers which may be used are lymphokines, such as interleukin (for example and without limitation, IL-1, -2, -3, -4, -5, and -6) or interferon (for example and without limitation, alpha, beta and gamma), erythropoietin, and colony stimulating factors (for example and without limitation, G-CSF, GM-CSF, and M-CSF). Peptide hormones include melanocyte stimulating hormone, follicle stimulating hormone, luteinizing hormone, and human growth hormone. Fibrinolytic enzymes include tissue-type plasminogen activator, streptokinase and urokinase. Serum proteins include human serum albumin and the lipoproteins.

Antibodies useful as targeting proteins may be polyclonal or monoclonal. A number of monoclonal antibodies (MAbs) that bind to a specific type of cell have been developed. These include MAbs specific for tumor-associated antigens in humans. Exemplary of the many MAbs that may be used are anti-TAC, or other interleukin-2 receptor antibodies; NR-ML-05, or other antibodies that bind to the 250 kilodalton human melanoma-associated proteoglycan; NR-LU-10, a pancarcinoma antibody directed to a 37-40 kilodalton pancarcinoma glycoprotein; and OVB3, which recognizes an as yet unidentified, tumor-associated antigen. Antibodies derived through genetic engineering or protein engineering may be used as well.

The antibody employed as a targeting agent in the present disclosure may be an intact molecule, a fragment thereof, or a functional equivalent thereof. Examples of antibody fragments useful in the compositions of the present disclosure are F(ab′)₂, Fab′ Fab and Fv fragments, which may be produced by conventional methods or by genetic or protein engineering.

In some embodiments, the oligonucleotide portion of the present invention may serve as an additional or auxiliary targeting moiety. The oligonucleotide portion may be selected or designed to assist in extracellular targeting, or to act as an intracellular targeting moiety. That is, the oligonucleotide portion may act as a DNA probe seeking out target cells. This additional targeting capability will serve to improve specificity in delivery of the composition to target cells. The oligonucleotide may additionally or alternatively be selected or designed to target the composition within target cells, while the targeting protein targets the conjugate extracellularly.

It is contemplated that the targeting moiety can, in various embodiments, be attached to the nanoparticle or a oligonucleotide. In aspects wherein the targeting moiety is a oligonucleotide, it is contemplated that it is attached to the nanoparticle, or is part of a oligonucleotide that is conjugated to a therapeutic agent. In further aspects, the targeting moiety is associated with the nanoparticle composition, and in other aspects the targeting moiety is administered before, concurrent with, or after the administration of a composition of the disclosure.

### Dosing and Pharmaceutical Formulations

The term “therapeutically effective amount” as used herein, refers to an amount of a therapeutic agent sufficient to treat, ameliorate, or prevent the identified disease or condition, or to exhibit a detectable therapeutic or inhibitory effect. The effect can be detected by, for example, an improvement in clinical condition, reduction in symptoms, or by any of the assays or clinical diagnostic tests described herein. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As described elsewhere herein, the therapeutic agents described herein may be formulated in pharmaceutical compositions with a pharmaceutically acceptable excipient, carrier, or diluent. The therapeutic agent or composition comprising the therapeutic agent can be administered by any route that permits treatment of the disease or condition. In one aspect, administration is oral administration. Additionally, the therapeutic agent or composition comprising the therapeutic agent is, in certain aspects, delivered to a patient using any standard route of administration, including parenterally, such as intravenously, intraperitoneally, intrapulmonary, subcutaneously or intramuscularly, intrathecally, transdermally, rectally, orally, nasally or by inhalation. The disclosure also includes, in some aspects, a method for increasing the intracellular retention time of a composition as described herein. The disclosure further includes, in some aspects, a method for affecting the biodistribution or cellular efflux of a composition as described herein.

Slow release formulations may also be prepared from the agents described herein in order to achieve a controlled release of the active agent in contact with the body fluids in the gastro intestinal tract, and to provide a substantial constant and effective level of the active agent in the blood plasma. A suitable form of ON-NPs of the disclosure may be embedded for this purpose in a polymer matrix of a biological degradable polymer, a water-soluble polymer or a mixture of both, and optionally suitable surfactants. Embedding can mean in this context the incorporation of nanoparticles in a matrix of polymers. Controlled release formulations are also obtained through encapsulation of dispersed nanoparticles or emulsified micro-droplets via known dispersion or emulsion coating technologies.

Administration may take the form of single dose administration, or the therapeutic agent of the embodiments can be administered over a period of time, either in divided doses or in a continuous-release formulation or administration method (e.g., a pump). However the therapeutic agents of the embodiments are administered to the subject, the amounts of therapeutic agent administered and the route of administration chosen should be selected to permit efficacious treatment of the disease condition.

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8. More particularly, the pharmaceutical compositions may comprise a therapeutically effective amount of at least one therapeutic agent as described herein, together with one or more pharmaceutically acceptable excipients. Optionally, the pharmaceutical compositions may comprise a combination of the therapeutic agents described herein, or may include a second active agent useful in the treatment or prevention of bacterial infection (e.g., anti-bacterial or anti-microbial agents).

Formulations, e.g., for parenteral or oral administration, are most typically solids, liquid solutions, emulsions or suspensions, while inhalable formulations for pulmonary administration are generally liquids or powders. Alternative pharmaceutical compositions may be formulated as syrups, creams, ointments, and tablets.

The term “pharmaceutically acceptable excipient” refers to an excipient for administration of a pharmaceutical agent, such as the therapeutic agents described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include without limitation antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, and pH buffering substances. Liposomes are also included within the definition of pharmaceutically acceptable excipients.

The pharmaceutical compositions described herein may be formulated in any form suitable for an intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules wherein the active agent is mixed with an inert solid diluent, for example celluloses, lactose, calcium phosphate or kaolin, or as soft gelatin capsules wherein the active agent is mixed with non-aqueous or oil medium, such as glycerin, propylene glycol, polyethylene glycol, peanut oil, liquid paraffin or olive oil.

In another embodiment, pharmaceutical compositions may be formulated as suspensions comprising a therapeutic agent of the embodiments in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents (e.g., sodium carboxymethylcellulose, methylcellulose, hydroxypropyl methylcellulose, sodium alginate, polyvinylpyrrolidone, gum tragacanth, gum acacia); dispersing or wetting agents (e.g., a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate)); and thickening agents (e.g., carbomer, beeswax, hard paraffin or cetyl alcohol). The suspensions may also contain one or more preservatives (e.g., acetic acid, methyl or n-propyl p-hydroxy-benzoate); one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using those suitable dispersing or wetting agents and suspending agents, including those mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

Also contemplated are therapeutic agents which have been modified by substitutions or additions of chemical or biochemical moieties which make them more suitable for delivery (for example and without limitation, to increase solubility, bioactivity, palatability, decrease adverse reactions), for example and without limitation by esterification, glycosylation, and PEGylation.

In some aspects, compositions are provided that further comprise a detectable marker. As used herein, a “detectable marker” is any label that can be used to identify the location of the composition, either in vivo or in vitro. Non-limiting examples of detectable markers are fluorophores, chemical or protein tags that enable the visualization of a polypeptide. Visualization may be done with the naked eye, or a device (for example and without limitation, a microscope) and may also involve an alternate light or energy source.

Combinations of therapeutic agents are also contemplated by the present disclosure, and they may, in various aspects, be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active agents sequentially, e.g., in separate solution, emulsion, suspension, tablets, pills or capsules, or by different injections in separate syringes. In general, during alternation therapy, an effective dosage of each active agent is administered sequentially, i.e., serially, whereas in simultaneous therapy, effective dosages of two or more active agent are administered together. Various sequences of intermittent combination therapy may also be used. Also contemplated by the present disclosure are embodiments wherein a therapeutic agent is associated with an additional oligonucleotide-functionalized nanoparticle. Further aspects include administration of a therapeutic agent that is not associated with a nanoparticle, and can freely traverse a cell membrane.

The invention will be more fully understood by reference to the following examples which detail exemplary embodiments of the invention. They should not, however, be construed as limiting the scope of the invention. All citations throughout the disclosure are hereby expressly incorporated by reference.

### EXAMPLES

### Example 1

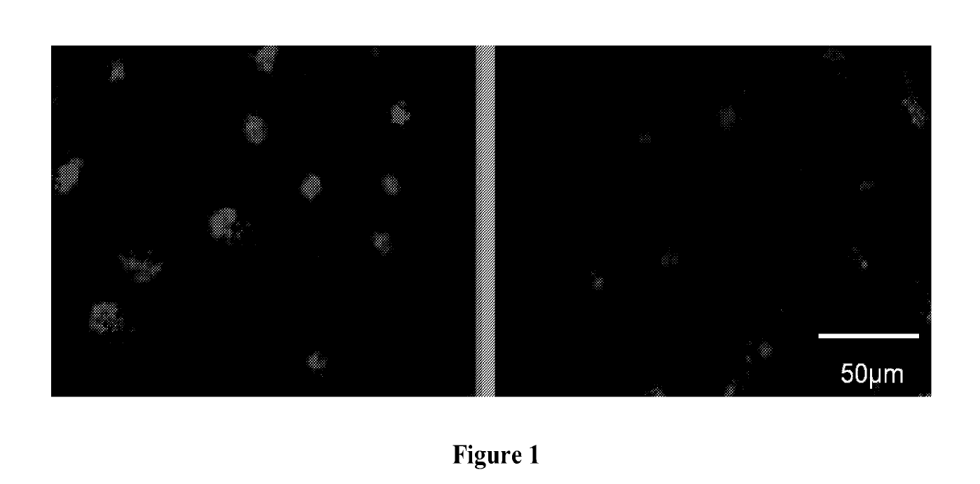

In this example, hydrophobic drug-like molecules (short thiolated polyethylene glycol (PEG) chains) were conjugated to a cyanine dye (Cy5) and adsorbed onto the surface of Au NPs along with thiolated DNA (PEG-Cy5-DNA Au NP conjugates). This created a co-monolayer of added molecules. In order to demonstrate the role of oligonucleotides in this strategy, either thiolated PEG alone, thioated oligonucleotides alone, or varying ratios of the molecules were used. The heterogeneous Au NPs were incubated in the presence of cells. Au NPs modified with DNA and RNA in combination with the PEG-cyanine dye showed strong intracellular florescence (PEG-Cy5-DNA), while the PEG-Cy5 modified Au NPs do not (FIG. 1). These studies showed that oligonucleotide modified Au NPs are capable of solubilizing and transporting hydrophobic drug-like molecules into cells.

### Example 2

In this example covalently bound Paclitaxel-DNA-gold nanoparticle (AuNP) conjugates were synthesized, characterized, and tested in vitro for drug delivery and biological activity. In addition, these conjugates were labeled with a fluorescent dye permitting imaging to confirm cell uptake and intracellular tracking. These nanoconjugates solve three common problems associated with paclitaxel as an effective chemotherapeutic agent: (1) enhanced solubility in aqueous systems such as buffers containing high concentration of salts and serum-containing cell culture medium; (2) increased therapeutic effect in paclitaxel-resistant cell lines; (3) providing a method for its detection and tracking.

All materials and solvents were purchased from Sigma-Aldrich Chemical Co. (St. Louis, Mo., USA) and used without further purification unless noted. Citrate-stabilized AuNPs (13±1.0 nm diameter) were prepared by the Frens method [Frens, Nature-Physical Science 241(105): 20-22 (1973)], resulting in approximately 10 nM solutions. Compound 1 was synthesized by succinic anhydride according to the literature [Deutsch ct al., J Med Chem, 32(4): 788-92 (1989)], adding a carboxyl acid group on the molecule at the C-2′-OH position as shown in Scheme 1 (Sequence shown in scheme 1 is SEQ ID NO: 2). The compound 1 was characterized by ESI-MS (Thermo Finnegan LCQ, Integrated Molecular Structure Education and Research Center, Northwestern University). M/Z: Calcd.=953.98. Found=953.92.

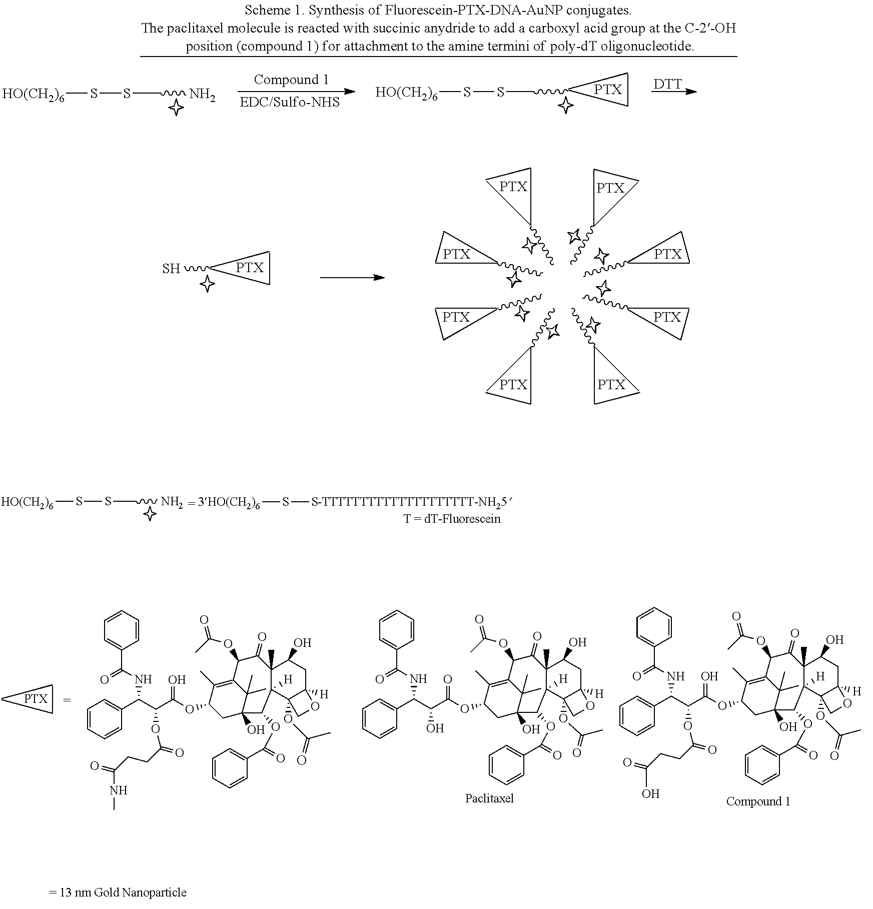

### General Cell Culture

MCF7, SKOV-3 and MES-SAIDx5 cells were purchased from American Type Culture Collection (ATCC, Manassas, Va., USA). Media, Dulbecco's phosphate buffered saline (DPBS), and 0.25% trypsinlEDTA were purchased from Invitrogen (Carlsbad, Calif., USA). MCF7 cells were cultured in Eagle's Minimum Essential Medium (EMEM) supplemented with 10% fetal bovine serum (FBS) and 0.01 mg/ml bovine insulin. SKOV-3 and MES-SA/Dx5 cells were cultured using McCoy's 5A modified media supplemented with 10% FBS. All experiments were performed in the aforementioned cell-specific media in a 5% CO₂ incubator at 37° C.

### Fluorescence Imaging

MCF7 and MES-SA/Dx5 cells were grown on Lab-Tek® II Chamber #1.5 German Coverglass System (Thermo Scientific—Nunc International, Naperville, Ill., USA) for 24 hours prior to imaging. 0.42 nM Fluorescein-PTX-DNA-AuNPs (corresponding to fluorescein labeled strands with a concentration of 25 nM) were then added directly to the cell culture media. After 6 hours of treatment, cells were rinsed with PBS and fresh media added. Live cells were stained with Cellular Lights™ Actin-RFP (Invitrogen) and DRAQ5 (Biostatus Ltd.) for cytoplasmic actin staining and nuclear staining, respectively, according to manufacturer's instructions. Images were acquired on a Zeiss LSM 510 inverted microscope (computer controlled using Zeiss Zen software). An Appochromat water immersion objective (40×, NA 1.2) was used for all measurements.

### Synthesis of Paclitaxel-Oligonucleotide Conjugates

Oligonucleotides were synthesized on an Expedite 8909 Nucleotide Synthesis System (ABI) using standard solid-phase phosphoramidite methodology. Bases and reagents were purchased from Glen Research (Sterling, Va., USA). The oligonucleotide used to functionalize the AuNPs was amine functionalized strand 5′—NH2-T20-hexyldisulfide-3′ (SEQ ID NO: 1). The oligonucleotide was purified by reverse-phase high performance liquid chromatography (RP-HPLC) and characterized by MALDI-MS (Bruker Apex III, Integrated Molecular Structure Education and Research Center, Northwestern University). The concentration of oligonucleotide was determined by monitoring the absorbance at 260 nm with a UV-Vis spectrophotometer. The strand was then reacted with compound 1 via EDC/Sulfo-NHS chemistry to prepare the PTX-DNA conjugate. In a typical reaction, 0.5 mL of compound 1 in acetonitrile solution was added to 1 mL of 10 times molar excess of Sulfo-NHS and EDC solution in HEPES buffer (0.1 M, pH=7). The resultant mixture was allowed to react at room temperature for 15 min. 0.5 molar equivalents (relative to compound 1) of oligonucleotide strand 5′—NH2-T20-hexyldisulfide-3′ (SEQ Ill NO: 1) was added to the solution. The reaction mixture was shaken gently for 3 days at room temperature. The PTX-DNA conjugate was purified by RP-HPLC and characterized by MALDI-MS. For quantification of paclitaxel loaded on the nanoparticle and cellular imaging, an additional Fluorescein/amine-modified strand (5′-NH2-T9-(Fluorescein-dT Phosphoramidite)-T10-hexyldisulfide 3′; SEQ ID NO: 2) was synthesized and reacted in a similar fashion to obtain a Fluorescein-labeled PTX-DNA conjugate.

Thus, nanoparticle conjugates were prepared by reacting citrate-stabilized gold nanoparticles with thiolated oligonucleotides containing a terminal paclitaxel (Scheme 1). First, as described above, DNA oligomers were synthesized on a solid support with a terminal amine group for covalent attachment to paclitaxel, which was modified by succinic anhydride through EDC/Sulfo-NHS coupling chemistry in order to add a carboxyl acid group on the molecule at the C-2′-OH position to farm compound 1. After purification by RP-HPLC, the paclitaxel-DNA (PTX-DNA) conjugates were characterized by matrix-assisted laser desorption/ionization mass spectrometry (MALDI-MS), which confirmed formation of the conjugates (Figure S1). The PTX-DNA conjugates were then immobilized on citrate-stabilized AuNPs in accordance to analogous literature procedures used to make DNA-AuNPs, ultimately yielding PTX-DNA-AuNPs [Hurst et al., Anal Chem 78(24): 8313-8 (2006)]. This method is described in more detail below.

### Preparation of PTX-DNA-AuNPs and Fluorescein-PTX-DNA-AuNPs

The oligonucleotide AuNP conjugates were synthesized as described previously [Hurst et al., Anal Chem 78(24): 8313-8 (2006)]. Briefly, disulfide functionalized oligonucleotides were freshly cleaved by dithiothreitol (DTT) for 1 hour at room temperature prior to use. The cleaved oligonucleotides were purified using NAP-10 columns (GE Healthcare). Freshly cleaved oligonucleotides were then added to gold nanoparticles (1OD/1 mL). After a 16 hour incubation, the concentrations of PBS and sodium dodecyl sulfate (SDS) were brought to 0.01M and 0.01%, respectively. The oligonucleotide/gold nanoparticle solution was allowed to incubate at room temperature for 20 minutes. NaCl was added using 2 M NaCl with repeated salting increments of 0.02 M NaCl every 5 hours until a concentration of 0.1 M NaCl was reached while maintaining an SDS concentration of 0.01%. The salting process was followed by an overnight incubation at room temperature. The final conjugates were stored in buffer with excess oligonucleotides at −4° C. Before use, the PTX-DNA-AuNP or Fluorescein-PTX-DNA-AuNP conjugates were spun down and washed until there were no strands detected by MALDI-MS in the supernatant.

Excess PTX-DNA was removed through repeated centrifugation and resuspension of PTX-DNA-AuNPs until no PTX-DNA was detected by MALDI-MS in the supernatant. Fluorescein-labeled PTX-DNA conjugates were synthesized as described in scheme 1 in order to produce Fluorescein-PTX-DNA-AuNPs for both imaging through confocal microscopy and subsequent loading of paclitaxel quantification onto the nanoparticle conjugates. In order to determine the number of paclitaxel molecules loaded on each particle, fluorescent PTX-DNA was chemically disassociated from the gold nanoparticle surface with dithiothreitol (DTT), and the concentrations of fluorescent PTX-DNA and nanoparticles measured as described previously [Hurst et al., Anal Chem 78(24): 8313-8 (2006)]. The amount of paclitaxel molecules per nanoparticle was determined to be 59±8 paclitaxel per nanoparticle conjugate by dividing the concentration of fluorescent oligonucleotides with the concentration of nanoparticles.

### Quantification of Alkanethiol Oligonucleotides Loaded on Gold Nanoparticles

The number of oligonucleotides loaded on each particle was determined by measuring the concentration of nanoparticles and the concentration of fluorescent DNA in each sample as previously reported [Hurst et al., Anal Chem 78(24): 8313-8 (2006)]. The concentration of gold nanoparticles in each aliquot was determined by performing UV-vis spectroscopy measurements. These absorbance values were then related to the nanoparticle concentration via Beefs law (A=εbc). The wavelength of the absorbance maximums (λ) and extinction coefficients (ε) used for 13 nm gold nanoparticles are as follows: λ=520 nm, ε=2.7×10⁸ M⁻¹cm⁻¹.

In order to determine the concentration of fluorescent oligonucleotides in each aliquot, DNA was chemically displaced from the nanoparticle surface using 1.0 M DTT in 0.18 M PBS, pH 8.0. The oligonucleotides were cleaved from the nanoparticle surface into solution during an overnight incubation, and the gold precipitate subsequently removed by centrifugation. To determine oligonucleotide concentration, 100 μL of supernatant was placed in a 96-well plate and the fluorescence was compared to a standard curve prepared with the same 1.0 M DTT buffer solution. During fluorescence measurements, the fluorophore was excited at 490 nm and the emission was collected at 520 nm.

The number of oligonucleotides per particle for each aliquot was calculated by dividing the concentration of fluorescent oligonucleotides by the concentration of nanoparticles. The experiment was repeated three times using fresh samples to obtain reliable error bars.

### Dynamic Light Scattering (DLS) and Transmission Electron Microscopy (TEM)

PTX-DNA-AuNPs or DNA-AuNPs were resuspended in 200 uL PBS buffer with oligonucleotide strands of an equivalent concentration of 25 μM. Hydrodynamic size measurements were conducted using the Zetasizer Nano ZS (Malvern, Worcestershire, U.K.). The size measurements were performed at 25° C. at a 173° scattering angle in disposable micro cuvettes (minimum volume 40 μL, Malvern, Worcestershire, U.K.). The mean hydrodynamic diameter was determined by cumulative analysis.

Transmission Electron Microscopy (TEM) was performed using a 200 kV Hitachi H-8100 TEM (EPIC, Northwestern University). Diluted PTX-DNA-AuNPs in deionized water were pipetted onto a commercial carbon TEM grid (Ted Pella Inc., Redding, Calif.). Upon air drying for 2 hours, samples were then observed within a Hitachi H-8100 TEM.

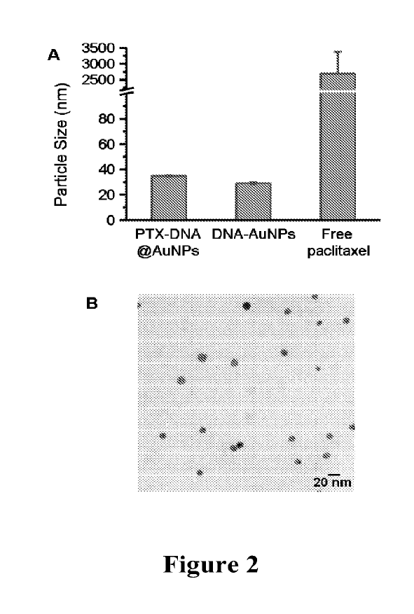

When suspended in aqueous solution, the PTX-DNA-AuNP conjugates appear as a clear deep red solution due to the Au plasmon resonance at 520 nm. The resulting conjugates are stable for months at 4° C., in stark contrast to unconjugated free paclitaxel in PBS, where the resultant suspension is turbid and a mass of pellets can be clearly observed. UV-Vis spectroscopy of the PTX-DNA-AuNPs surface Plasmon band confirmed the absence of particle aggregation after drug conjugation. Furthermore, it is interesting to note that the resultant drug-nanoparticle conjugates exhibit significantly enhanced hydrophilicity and solubility in salt-containing buffer. Dynamic light scattering (DLS) analysis and TEM images (FIG. 2) indicate that PTX-DNA-AuNPs containing 25 μM paclitaxel is well dispersed in PBS with a narrow-size distribution, whereas severe aggregation occurs when the same amount of hydrophobic paclitaxel is suspended even after sonication for several seconds in PBS. In comparison with free paclitaxel (0.4 μg/mL) [Hwu et al., J Am Chem Soc 131 (1), 66-8 (2009); Skwarczynski et al., Journal of Medicinal Chemistry 49(25): 7253-7269 (2006)], conjugated PTX-DNA-AuNPs enhance the solubility of paclitaxel from 0.4 μg/mL to above 21.35 μg/mL (corresponding to 25 μM paclitaxel), an increased factor of at least 53. When compared with unmodified DNA-AuNPs (29.2 E 0.6 nm), PTX-DNA-AuNPs exhibit a slightly larger average size of 34.7±1.7 nm with a polydiversity index (PDI) of 0.2.

It was demonstrated that the therapeutic effects of drug-loaded nanoparticles would depend on successful internalization and sustained retention by diseased cells [Zhang et al., Acta Biomater 6(6): 2045-52; Jin et al., Biomaterials 28(25): 3724-30 (2007)]. In this work, DNA-AuNPs were selected as a delivery vehicle for paclitaxel specifically due to the ability of DNA-AuNPs to enter cells efficiently [Giljohann et al., Angew Chem Int Ed Engl 49(19): 3280-94 (2010)]. Moreover, DNA-AuNPs show a superior capacity of cellular uptake when compared to other types of AuNPs. For example, HeLa cells internalize only a few thousand citrate-coated gold nanoparticles [Chithrani et al., Nano Lett 6(4): 662-8 (2006)], compared to over one million DNA-AuNPs under nearly identical conditions [Giljohann et al., Nano Lett 7(12): 3818-21 (2007)].

The ability of Fluorescein-PTX-DNA-AuNPs to enter cells was investigated by confocal microscopy using gold nanoparticles functionalized with a monolayer of Fluorescein-labeled PTX-DNA molecules. Confocal fluorescence images showed the successful internalization of the fluorescently labeled conjugates in MCF7 human breast adenocarcinoma cells and MES-SA/Dx5 human uterine sarcoma cells after 6 hours of incubation. Within MES-SA/Dx5 cells, most Fluorescein-PTX-DNA-AuNPs are observed in the cytoplasm, indicating the efficient translocation of the paclitaxel-gold nanoparticle conjugates. Within MCF7 cells, some nanoparticles are colocalized within the cytoplasm, while others are located in small vesicles in the perinucleur region.

### TUNEL Assay

MCF7 and MES-SA/Dx5 cells were seeded on 0.17 mm thick coverslips in 12-well plates at a density of 2×10⁵ cells/well for 24 hours prior to fluorescent TUNEL assay. Cells were treated with nothing, DNA-AuNPs at a DNA strand concentration of 100 nM (negative controls), 100 nM of free paclitaxel and compound 1 (positive controls), PTX-DNA-AuNPs at the equivalent paclitaxel concentrations of 50 nM and 100 nM (samples), respectively, for 48 hours. Live cells were rinsed and stained in accordance with instructions and materials for adherent cultured cells provided by Chemicon International ApopTag Plus Fluorescein In situ Apoptosis Detection Kit 57111 (Temecula, Calif.). ApopTag utilizes the terminal deoxynucleotidyl transferase (TdT) enzyme to amplify the fluorescein-conjugated anti-digoxigenin antibody, a secondary antibody towards digoxigenin-labeled nucleotide-labeled 30-OH termini on DNA fragments. Images were acquired on a Zeiss LSM 510 inverted microscope (computer controlled using Zeiss Zen software).

### MTT Assay

The cytotoxicity profiles of PTX-DNA-AuNP conjugates, paclitaxel and compound 1 in MCF7, MES-SA/Dx5 and SKOV-3 cells were investigated using a 3-(4,5-dimethylthiazol-2-yl)-2,5-diphenyltetrazolium bromide (MTT) assay following the manufacturer's protocol. Briefly, cells were seeded on 96-well plates for 24 hours before the assay at a density of 1.5×10⁴ cells/well. Following 24 hours of growth, media was replaced with 200 μL of corresponding sample solutions, which were freshly prepared at varying concentrations in complete cell culture media. Cells in media containing 10% FBS with nothing added were used as controls. After 12 hours or 48 hours of treatment, cells were rinsed and cultured with fresh medium containing 0.5 mg/mL of MTT for an additional 3 hours. Following careful aspiration of MTT solution and media after MTT incubation, 200 μL of MTT solubilization solution was added to each well and thoroughly mixed. The optical density at 570 mm was measured using a Safire microplate reader (Tecan Systems, Inc., San Jose, Calif.). Background absorbance at 690 nm was subtracted. Values were expressed as a percentage of the control (incubated with media alone). All conditions were done in sextuplicate in two independent experiments for each cell line.

In order to test the preserved activity of the drug present on the nanoparticle conjugate surface, a terminal deoxynucleotidyl transferase dUTP nick end labeling (TUNEL) assay [Gavrieli et al., J. Cell Biol. 119(3): 493-501 (1992)] was performed to detect DNA fragmentation and apoptosis induced by paclitaxel. MCF7 or MES-SA/Dx5 cells were incubated with drug-free DNA-AuNPs, free paclitaxel, compound 1 and PTX-DNA@AuNPs, respectively, at varying concentrations for 48 hours. Unlike MCF7 cells, MES-SAIDx5 cells express high levels of mdr-1 mRNA and P-glycoprotein and exhibit a marked cross resistance to a number of chemotherapeutic agents including paclitaxel [Angelini et al., Oncol Rep 20(4): 731-5 (2008); Chen et al., Br J Cancer 83(7): 892-8 (2000); Chu et al., Toxicol Lett 181(1): 7-12 (2008)]. Untreated cells and drug-free DNA-AuNPs were used as negative controls, showing minimal sign of apoptosis and the greatest cell viability. When treated with 100 nM of free paclitaxel or compound 1, MES-SA/Dx5 cells exhibit a lower fraction of TUNEL-positive signals in comparison with MCF7 cells, demonstrating the MES-SA/Dx5 cells' inherent resistance towards paclitaxel. It is worthy to note that the intense signal of TUNEL-positive cells and diminished population relative to positive controls are clearly observed in both MCF7 cells and MES-SA/Dx5 cells as well after incubation with PTX-DNA@AuNP conjugates containing 100 nM of paclitaxel. The TUNEL staining images indicate that paclitaxel remains active upon conjugation, strongly suggesting the resulting gold nanoparticle conjugates have the potential to circumvent paclitaxel resistance.

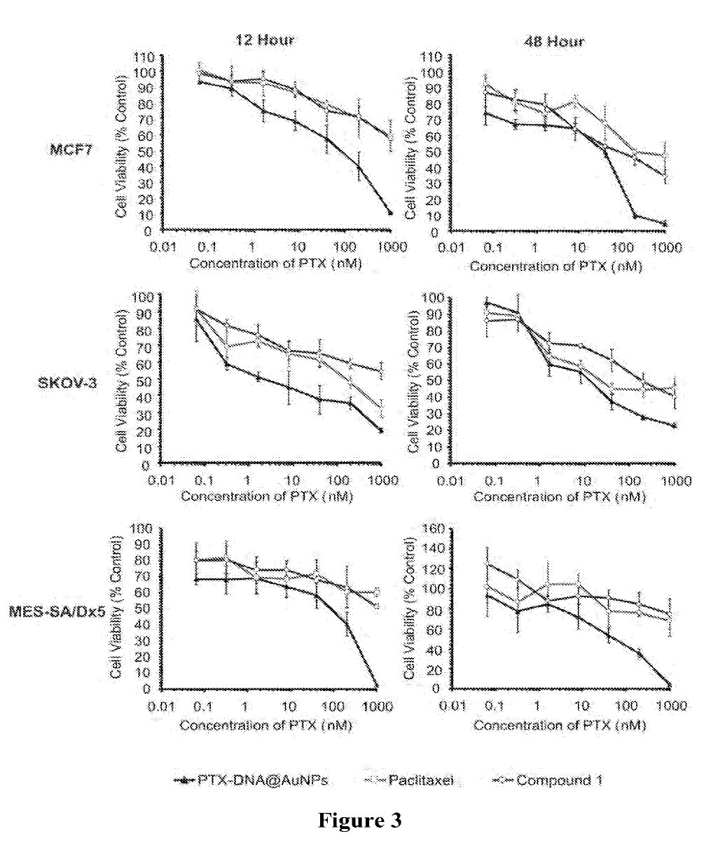

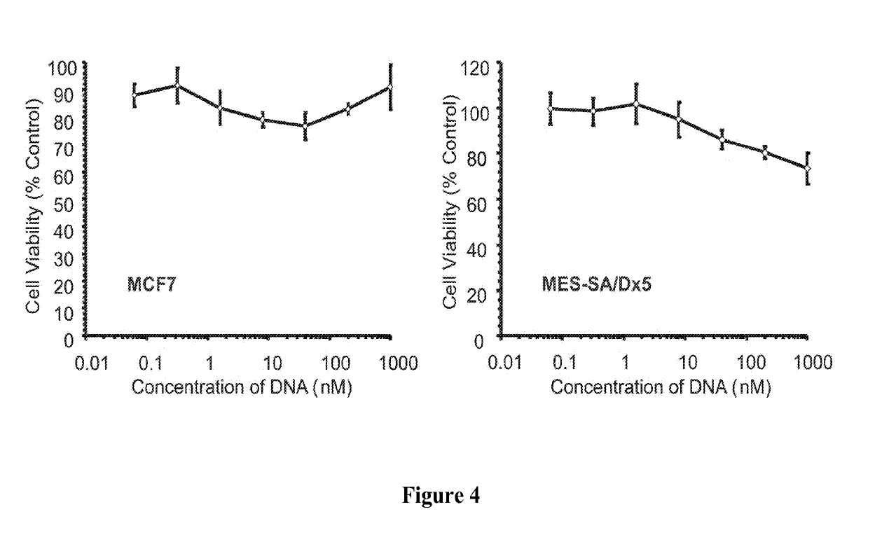

In order to evaluate the efficiency of PTX-DNA@AuNPs, their ability to induce death within cancer cells of various origins was investigated. FIG. 3 shows the in vitro viability of MCF7, MES-SA/Dx5 and SKOV-3 ovarian cancer cells cultured with paclitaxel, compound 1 and PTX-DNA-AuNP conjugates at varying equivalent paclitaxel concentrations ranging from 0.064 to 1000 nM. MTT assays of DNA-AuNPs containing equivalent DNA strand concentrations were also conducted in MCF7 and MES-SA/Dx5 cells as negative controls (FIG. 4). DNA-AuNPs without drug generate little to no cytotoxic profiles within MCF7 and MES-SA/Dx5 cells even after 48 hours incubation. More than 75-90% of cells are viable at 48 hours when cultured with DNA-AuNPs at DNA concentrations at or above 1 μM. However, as shown in FIG. 3, after 12 hours or 48 hours treatment with different concentrations of PTX-DNA-AuNPs, cytotoxicity was observed in all three cell lines as compared with paclitaxel and compound 1 alone. In particular, MES-SA/Dx5 cell viability after 2 days incubation at 200 nM drug concentration was decreased from 84.3% for compound 1 to 76.0% for paclitaxel alone and 35.4% for the PTX-DNA@AuNP formulation. Both paclitaxel and compound 1 did not display any significant therapeutic activity under the same conditions in paclitaxel-resistant MES-SA/Dx5 cells, while the activity of paclitaxel was considerably enhanced when tethered to DNA-AuNPs. Similarly, in MCF7 and SKOV-3 cells, PTX-DNA-AuNPs reflect efficacy greater than that of paclitaxel and compound 1 after 12 hour and 48 hour incubation. The improved cytotoxicity of PTX-DNA-AuNPs could be attributed to the enhanced hydrophilicity as well as the increased cellular uptake of the conjugates in comparison with free drug.

The effect in MCF7, SKOV-3 and MES-SA/Dx5 cells after incubation at various drug concentrations are summarized by their IC₅₀ values (Table 1). The data demonstrate the advantage of utilizing nanoparticle conjugates in relation to free drugs. For instance, the IC₅₀ value for MCF7 cells decreases from above 1 μM and 193 nM for free paclitaxel to 119.4 nM and 52.6 nM for PTX-DNA-AuNPs after 12 hour and 48 hour incubation, respectively. In resistant MES-SA/Dx5 cells, both paclitaxel and compound 1 have IC₅₀ values above 1 μM, whereas PTX-DNA-AuNPs exhibit IC₅₀ values of 118 nM and 104.5 nM after incubation for 12 hours and 48 hours, respectively. A similar trend is observed in SKOV-3 cells. After 48 hour incubation, PTX-DNA-AuNPs have an IC₅₀ value of 17.5 nM, lower than that of paclitaxel (28.9 nM) and compound 1 (188.0 nM), attesting to the enhanced activity across different cancerous cell lines of the paclitaxel compound upon conjugation to a gold nanoparticle via a DNA linker.

TABLE 1
IC₅₀ of PTX-DNA-AuNPs, paclitaxel and compound 1 after 12 hour and
48 hour incubation in MCF7, SKOV-3 and MES-SA/Dx5 cells.

| | | IC₅₀ (nM Paclitaxel) | | |
|---|---|---|---|---|
| | | PTX-DNA | | |
| | Incubation time (h) | @AuNPs | Paclitaxel | Compound1 |
| MCF7 | 12 | 119.4 | >1000 | >1000 |
| | 48 | 52.6 | 193.0 | 133.2 |
| SKOV-3 | 12 | 4.3 | 175.6 | >1000 |
| | 48 | 17.5 | 28.9 | 188.0 |
| MES-SA/ | 12 | 118.0 | >1000 | >1000 |
| Dx5 | 48 | 104.5 | >1000 | >1000 |

Utilizing the inherent surface chemistry of gold nanoparticles, several important features pertinent to DNA-AuNP based drug delivery can be ascertained. In this study, an efficient strategy for delivering hydrophobic paclitaxel while simultaneously overcoming drug efflux in human cancer cells was shown. PTX-DNA-AuNPs were fabricated by covalently attaching hydrophobic paclitaxel onto gold nanoparticles via a DNA spacer, which resulted in significantly enhanced hydrophilicity and stability in PBS as compared to free paclitaxel alone. The visualization of fluorescein labeled PTX-DNA-AuNPs within human breast adenocarcinoma cells and uterine sarcoma cells by confocal fluorescence microscopy demonstrates the efficient cellular internalization, delivery and distribution of paclitaxel. Furthermore, the therapeutic activity of paclitaxel was enhanced in vitro against several cancer cell lines when attached onto DNA-AuNPs. In TUNEL and MTT assays across several concentrations and cell lines, PTX-DNA-AuNPs were more effective than free drug in inducing apoptosis most notably within paclitaxel resistant MES-SA/Dx5 cells.

What is claimed:

1. A drug delivery composition comprising an oligonucleotide-modified nanoparticle and a therapeutic agent, said therapeutic agent being deliverable at a significantly lower level in the absence of attachment to the oligonucleotide-modified nanoparticle compared to the delivery of the therapeutic agent when attached to the oligonucleotide-modified nanoparticle, wherein the composition has a number of oligonucleotide molecules compared to therapeutic agent molecules in a ratio that is sufficient to allow transport of the therapeutic agent into a cell.

2. The composition of claim 1 wherein the therapeutic agent is a low molecular weight therapeutic agent.

3. The composition of claim 1 or claim 2 wherein the therapeutic agent is hydrophobic.

4. The composition of any of claims 1 through 3 wherein the therapeutic agent is hydrophilic.

5. The composition of any one of claims 1 through 4, further comprising a detectable marker.

6. The composition of any of claims 1 through 5 wherein the therapeutic agent is an agent selected from Table 2.

7. The composition of any one of claims 1 through 6 wherein the oligonucleotide and the therapeutic agent are independently directly attached to the nanoparticle.

8. The composition of any of claims 1 through 6 wherein the therapeutic agent is attached to the oligonucleotide attached to the nanoparticle.

9. The composition of claim 8 wherein the therapeutic agent is covalently attached to the oligonucleotide attached to the nanoparticle.

10. The composition of claim 8 wherein the therapeutic agent is non-covalently attached to the oligonucleotide attached to the nanoparticle.

11. The composition of any one of claims 1-10 wherein the ratio is a number comparison of oligonucleotide to therapeutic agent.

12. The composition of claim 11 wherein the ratio of the oligonucleotide to the therapeutic agent on a surface of the nanoparticle is at least about 1 oligonucleotide molecule:2 therapeutic agent molecules.

13. The composition of any one of claims 1 through 12, further comprising an additional therapeutic agent.

14. The composition of any one of claims 1 through 13 wherein the additional therapeutic agent is attached to the oligonucleotide-modified nanoparticle.

15. The composition of any one of claims 1 through 14 wherein the additional therapeutic agent is attached to a second oligonucleotide-modified nanoparticle.

16. The composition of any one of claims 1 through 15 wherein the additional therapeutic agent is not attached to the oligonucleotide-modified nanoparticle and freely traverses a cell membrane.

17. A method of treating a disease comprising the step of administering to a mammal a therapeutically effective amount of the composition of any of claims 1-16.

18. A kit comprising the composition of any of claims 1-16.


SEQUENCE LISTING (2 sequences)

SEQ ID NO: 1 - DNA, 20 bp, Artificial Sequence
  Synthetic oligonucleotide
tttttttttttttttttttt

SEQ ID NO: 2 - DNA, 19 bp, Artificial Sequence
  Synthetic oligonucleotide
ttttttttttttttttttt